SEQUENCE LISTING (20 sequences)

SEQ ID NO: 1 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 2 - protein, 605 aa, Homo sapiens
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 3 - DNA, 20 bp, Artificial Sequence
  synthetic nucleic acid
gttatggttctcacagatga

SEQ ID NO: 4 - DNA, 20 bp, Artificial Sequence
  synthetic nucleic acid
tactcatccatgtgaccatg

SEQ ID NO: 5 - DNA, 20 bp, Artificial Sequence
  synthetic nucleic acid
agatgggagctcacactgtg

SEQ ID NO: 6 - DNA, 20 bp, Artificial Sequence
  synthetic nucleic acid
cagcatgttcctatatgcag

SEQ ID NO: 7 - DNA, 20 bp, Artificial Sequence
  synthetic nucleic acid
cttgtacctgaaagccttgg

SEQ ID NO: 8 - DNA, 20 bp, Artificial Sequence
  synthetic nucleic acid
aagtgcacggtcaccaacag

SEQ ID NO: 9 - DNA, 20 bp, Artificial Sequence
  synthetic nucleic acid
atgtgggatgggtgctggat

SEQ ID NO: 10 - DNA, 20 bp, Artificial Sequence
  synthetic nucleic acid
gggggatgtccatgtgggat

SEQ ID NO: 11 - DNA, 20 bp, Artificial Sequence
  synthetic nucleic acid
gatggccttgccataggctg

SEQ ID NO: 12 - DNA, 20 bp, Artificial Sequence
  synthetic nucleic acid
actgaccagatatgactgtg

SEQ ID NO: 13 - DNA, 20 bp, Artificial Sequence
  synthetic nucleic acid
aaggccactgcttggtgacc

SEQ ID NO: 14 - DNA, 20 bp, Artificial Sequence
  synthetic nucleic acid
agcgtcctgccattggcttg

SEQ ID NO: 15 - DNA, 20 bp, Artificial Sequence
  synthetic nucleic acid
gcgggagcagggcatggagg

SEQ ID NO: 16 - DNA, 20 bp, Artificial Sequence
  synthetic nucleic acid
ccgacagtcgctcagctacc

SEQ ID NO: 17 - DNA, 20 bp, Artificial Sequence
  synthetic nucleic acid
acagtcgctcagctacctgg

SEQ ID NO: 18 - DNA, 20 bp, Artificial Sequence
  synthetic nucleic acid
gaggacacacttctcgccca

SEQ ID NO: 19 - DNA, 20 bp, Artificial Sequence
  synthetic nucleic acid
gaccaggtagtccttgcagc

SEQ ID NO: 20 - DNA, 20 bp, Artificial Sequence
  synthetic nucleic acid
ccaggtagctgagcgactgt

(19) United States
(12) Patent Application Publication
(10) Pub. No.: US 2023/0070477 A1
(43) Pub. Date: Mar. 9, 2023

(54) REPROGRAMMING THE METABOLOME TO DELAY ONSET OR TREAT NEURODEGENERATION

(71) Applicant: THE TRUSTEES OF CULUMBIA UNIVERSITY IN THE CITY OF NEW YORK, New York, NY (US)

(72) Inventors: Stephen H. Tsang, New York, NY (US); Xuan Cui, New York, NY (US); Nan-Kai Wang, New York, NY (US); Pei-Kang Liu, New York, NY (US)

(21) Appl. No.: 17/760,152

(22) PCT Filed: Feb. 8, 2021
(86) PCT No.: PCT/US2021/017077

§ 371 Date: Aug. 4, 2022

Related U.S. Application Data
(60) Provisional application No. 62/971,370, filed on Feb. 7, 2020.

(51) Int. Cl. C12N 15/86; A61K 48/00; C12N 15/11

(52) U.S. Cl. CPC C12N 15/86; A61K 48/005; C12N 15/111; C12N 2830/008; C12N 2310/14; C12N 2750/14143

(57) ABSTRACT

The present disclosure relates to methods and compounds for reprogramming metabolism in one specific retinal and neuronal cell type leading to improved cell and tissue survival and function. In particular, the present disclosure relates to increasing PGC1α/Pgc1α or NRF2/Nrf2 or inhibiting HIF/Hif or KEAP1/Keap1 to reprogram metabolism and survival of cells in a variety of neurodegenerative conditions, and specifically those which cause blindness.

### CROSS-REFERENCE TO RELATED APPLICATIONS

The present application claims priority to U.S. Provisional Patent Application No. 62/971,370 filed Feb. 7, 2020, which is incorporated herein by reference in its entirety.

### GOVERNMENT SUPPORT

This invention was made with government support under U54OD020351, R24EY028758, R01AR059703, R24EY027285, R01EY018213, R01EY024698, R01EY026682, U01EY030580, and R21AG050437 awarded by the National Institutes of Health. The government has certain rights in the invention.

### FIELD

The present disclosure relates to methods and compounds for reprogramming metabolism in one specific retinal and neuronal cell type leading to improved tissue survival and function. In particular, the present disclosure relates to increasing PGC1α/Pgc1α or NRF2/Nrf2 or inhibiting HIF/Hif or KEAP1/Keap1 to reprogram metabolism and survival of cells and tissues, in a variety of neurodegenerative conditions, and specifically those which cause blindness.

### BACKGROUND

Neurodegenerative diseases causing blindness have limited treatment options. These diseases include retinitis pigmentosa, glaucoma, age-related macular degeneration, and autosomal dominant optic atrophy.

Retinitis pigmentosa (RP) is the most common inherited retinal dystrophy, caused by greater than 71 mutations that primarily cause rod photoreceptor death. Although cones are not killed directly by the mutations responsible for RP, cone death always follows rod death (Campochiaro and Mir. 2018) and starts after the major rod death phase (Punzo, et al. 2009). Recent advances using retinal prostheses (da Cruz, et al. 2016) or RPE65 gene therapy (Russell, et al. 2017) provide respective treatment options for patients with end-stage RP or those carrying the RPE65 mutation. Clinical gene therapy trials for RP that focus on augmentation or repair of a single gene, even if successful, would only be applicable to patients carrying that specific gene mutation (Takahashi, et al. 2018). Thus, a more generally applicable therapy targeting a pathway common to all RP could serve as potential treatment for RP caused by different rod-specific mutations (Duncan, et al. 2018)

Glaucoma affects more than 70 million people and is the most common neurodegenerative disease causing blindness worldwide (Quigley and Broman 2006). Retina ganglion cell (RGC) degeneration and death are common hallmarks of neurodegenerative diseases causing blindness, such as glaucoma and autosomal dominant optic atrophy (ADOA). Although drugs can lower intraocular pressure and decrease the rate of RGC death, these treatments do not offer a cure. Again, even if successful, clinical gene therapy trials that focus on augmentation or repair of a single gene would only be applicable to patients carrying that specific gene mutation (Lam, et al. 2010). Thus, a more generally applicable therapy targeting a common pathway in RGC death could serve as a potential treatment for patients with glaucoma and optic atrophy.

Thus, there is an urgent need for additional therapeutics as well as more broadly effective gene therapies for alleviating retinal degenerative diseases such as RP, and more broadly for promoting neuronal survival in neurodegenerative diseases such as glaucoma,

Alzheimer's, Parkinson's disease, Huntington's disease, Amyotrophic lateral sclerosis (ALS), Lewy body dementia, and similar neurodegenerative conditions or other conditions that would benefit from upregulating anabolism and downregulating catabolism to promote neuronal survival.

### SUMMARY

The present disclosure provides for a non-gene-specific strategy for treating, preventing and/or delaying the onset of all retinitis pigmentosa (RP), glaucoma, and autosomal dominant optic atrophy (ADOA) patients, regardless of their genetic background, by restoring the metabolic balance which in turn can prevent photoreceptor death and vision loss. The present disclosure provides for compositions and methods for overexpressing or increasing proliferator-activated receptor gamma, coactivator 1 alpha (PGC1α) or nuclear factor erythroid 2-related factor (NRF2), or for inhibiting, ablating or decreasing hypoxia-inducible factor (HIF) or Kelch-like ECH-associated protein 1 (KEAP1), in order to delay the onset of, cure, treat or prevent retinal degenerative diseases causing blindness, such as retinitis pigmentosa (RP), age-related macular degeneration (AMD), autosomal dominant optic atrophy (ADOA) and glaucoma, as well as neurodegenerative diseases such as Alzheimer's disease (AD), Parkinson's disease (PD), Huntington's disease, Amyotrophic lateral sclerosis (ALS), and Lewy body dementia.

For prophylaxis, the present compositions can be administered to a subject in order to prevent the onset of one or more symptoms of neurodegenerative disease, including those which cause blindness. In one embodiment, the subject can be asymptomatic.

The present compositions may be used in vitro or administered to a subject. The administration may be topical, intravenous, intranasal, or any other suitable route as described herein. The present compositions may be administered by intravitreal injection, subretinal injection or suprachoroidal injection.

Using the compositions and methods of the disclosure, a subject (e.g., a mammalian subject, such as a human subject) that has or is at risk of developing a disease described herein may be administered a composition containing a transgene encoding one or more of proteins described herein. The composition may comprise a vector, for example, a viral vector, such as an adeno-associated virus (AAV) vector. In some embodiments, the transgene encodes PGC 1 α. In some embodiments, the transgene encodes NRF2.

Additionally, using the compositions and methods of the disclosure, a subject (e.g., a mammalian subject, such as a human subject) that has or is at risk of developing a disease described herein may be administered a composition containing a nucleic acid inhibitor or a nucleic acid encoding an inhibitor of one or more of proteins or a nucleic acid targeting one or more of the genes encoding the proteins described herein. The composition may comprise a vector, for example, a viral vector, such as an adeno-associated virus (AAV) vector or a lentivector. In some embodiments, the inhibitor is to HIF. In some embodiments, the inhibitor is to KEAP1.

The present disclosure provides a method of delaying the onset of, treating, preventing and/or curing a neurodegenerative disease, comprising administering to a subject in need thereof a therapeutically effective amount of an agent which increases PGC1α.

In a further embodiment, the present disclosure provides a method of delaying the onset of, treating, preventing and/or curing a neurodegenerative disease, comprising administering to a subject in need thereof a therapeutically effective amount of an agent which increases NRF2.

In some embodiments, the agents which increase PGC 1 α and NRF2 are nucleic acids which encode PGC1α and NRF2.

The present disclosure also provides a method of delaying the onset of, treating, preventing and/or curing a neurodegenerative disease, comprising administering to a subject in need thereof a therapeutically effective amount of a viral vector comprising nucleic acid which encodes PGC1α.

The present disclosure also provides a method of delaying the onset of, treating, preventing and/or curing a neurodegenerative disease, comprising administering to a subject in need thereof a therapeutically effective amount of a viral vector comprising nucleic acid which encodes NRF2.

The present disclosure also provides a method of delaying the onset of, treating, preventing and/or curing a neurodegenerative disease, comprising administering to a subject in need thereof a therapeutically effective amount of a composition which increases PGC 1α.

In a further embodiment, the present disclosure provides a method of delaying the onset of, treating, preventing and/or curing a neurodegenerative disease, comprising administering to a subject in need thereof a therapeutically effective amount of a composition which increases NRF2.

In some embodiments, the compositions comprise nucleic acids which encode PGC1α and/or NRF2. In some embodiments, the compositions comprise viral vectors comprising nucleic acids which encode PGC1α and/or NRF2.

In some embodiments, the viral vectors are recombinant or rAAV. In some embodiments, the rAAV are AAV2 serotype. In some embodiments, the viral vectors are AAV8 serotype.

In some embodiments, the viral vectors comprise promoters which target particular cells. In some embodiments, these targeted cells are retinal epithelial cells (RPE). In some embodiments, the targeted cells are retinal ganglion cells (RGC).

In certain embodiments, the present disclosure provides for a method of delaying the onset of, treating, preventing and/or curing a neurodegenerative disease, comprising administering to a subject in need thereof a therapeutically effective amount of a recombinant adeno-associated viral (AAV) vector comprising a transgene encoding PGC1α.

In certain embodiments, the present disclosure provides for a method of delaying the onset of, treating, preventing and/or curing a neurodegenerative disease, comprising administering to a subject in need thereof a therapeutically effective amount of a recombinant adeno-associated viral (AAV) vector comprising a transgene encoding NRF2.

In some embodiments, the rAAV are AAV2 serotype. In some embodiments, the viral vectors are AAV8 serotype.

In some embodiments, the viral vectors comprise promoters which target particular cells. In some embodiments, these targeted cells are retinal epithelial cells (RPE). In some embodiments, the targeted cells are retinal ganglion cells (RGC).

Also provided for herein is a method of method of delaying the onset of, treating, preventing and/or curing a neurodegenerative disease, comprising administering to a subject in need thereof a therapeutically effective amount of an agent which inhibits HIF or KEAP1. In some embodiments, agent is chosen from the group of proteins, nucleic acids, small molecules, chemicals and combinations thereof. In some embodiments, the nucleic acid is selected from the group consisting of an antisense oligonucleotide, a small interfering RNA (siRNA), a short hairpin RNA (shRNA), a guide RNA (gRNA), aptamer, and combinations thereof.

The present disclosure also provides a method of delaying the onset of, treating, preventing and/or curing a neurodegenerative disease, comprising administering to a subject in need thereof a therapeutically effective amount of a viral vector comprising nucleic acids which inhibit HIF or encode for nucleic acids which inhibit HIF or target HIF.

The present disclosure also provides a method of delaying the onset of, treating, preventing and/or curing a neurodegenerative disease, comprising administering to a subject in need thereof a therapeutically effective amount of a viral vector comprising nucleic acids which inhibit KEAP1 or encode for nucleic acids which inhibit KEAP1 or target KEAP1.

In a further embodiment, the present disclosure provides a method of delaying the onset of, treating, preventing and/or curing a neurodegenerative disease, comprising administering to a subject in need thereof a therapeutically effective amount of a composition which inhibits HIF or targets HIF.

In some embodiments, the compositions comprise nucleic acids which inhibit HIF or encode nucleic acids which inhibit HIF or target HIF. In some embodiments, the compositions comprise viral vectors comprising nucleic acids which inhibit HIF or encode nucleic acids which inhibit HIF or target HIF.

In a further embodiment, the present disclosure provides a method of delaying the onset of, treating, preventing and/or curing a neurodegenerative disease, comprising administering to a subject in need thereof a therapeutically effective amount of a composition which inhibits KEAP1 or targets KEAP1.

In some embodiments, the compositions comprise nucleic acids which inhibit or encode nucleic acids which inhibit KEAP1 or target KEAP1. In some embodiments, the compositions comprise viral vectors comprising nucleic acids which inhibit KEAP1 or encode nucleic acids which inhibit KEAP1 or target KEAP1.

In some embodiments, the viral vectors are recombinant or rAAV. In some embodiments, the rAAV are AAV2 serotype. In some embodiments, the viral vectors are AAV8 serotype.

In some embodiments, the viral vectors are lentiviral vectors.

In some embodiments, the viral vectors comprise promoters which target particular cells. In some embodiments, these targeted cells are retinal epithelial cells (RPE). In some embodiments, the targeted cells are retinal ganglion cells (RGC).

In certain embodiments, the present disclosure provides for a method of delaying the onset of, treating, preventing and/or curing a neurodegenerative disease, comprising administering to a subject in need thereof a therapeutically effective amount of a recombinant adeno-associated viral (AAV) vector comprising a nucleic acid which inhibits HIF or targets HIF.

In certain embodiments, the present disclosure provides for a method of delaying the onset of, treating, preventing and/or curing a neurodegenerative disease, comprising administering to a subject in need thereof a therapeutically effective amount of a lentiviral vector comprising a nucleic acid which inhibits HIF or targets HIF.

In some embodiments, the lentiviral vector is EIAV.

In some embodiments, the viral vectors comprise promoters which target particular cells. In some embodiments, these targeted cells are retinal epithelial cells (RPE). In some embodiments, the targeted cells are retinal ganglion cells (RGC).

In certain embodiments, the present disclosure provides for a method of delaying the onset of, treating, preventing and/or curing a neurodegenerative disease, comprising administering to a subject in need thereof a therapeutically effective amount of a recombinant adeno-associated viral (AAV) vector comprising a nucleic acid which inhibits KEAP1 or targets KEAP1.

In certain embodiments, the present disclosure provides for a method of delaying the onset of, treating, preventing and/or curing a neurodegenerative disease, comprising administering to a subject in need thereof a therapeutically effective amount of a lentiviral vector comprising a nucleic acid which inhibits KEAP1 or targets KEAP1.

In some embodiments, the lentiviral vector is EIAV.

In some embodiments, the viral vectors comprise promoters which target particular cells. In some embodiments, these targeted cells are retinal epithelial cells (RPE). In some embodiments, the targeted cells are retinal ganglion cells (RGC).

In certain embodiments, the present disclosure provides for a method of delaying the onset of, treating, preventing and/or curing a neurodegenerative disease, comprising administering to a subject in need thereof a therapeutically effective amount of a) a first nucleic acid sequence(s) encoding at least one guide RNA that hybridizes to the endogenous HIF gene in the patient, and a second nucleic acid sequence encoding a Cas nuclease, wherein the Cas nuclease cleaves the endogenous HIF gene creating a HIF knockout of the endogenous HIF gene in the subject.

In certain embodiments, the present disclosure provides for a method of delaying the onset of, treating, preventing and/or curing a neurodegenerative disease, comprising administering to a subject in need thereof a therapeutically effective amount of a) a first nucleic acid sequence(s) encoding at least one guide RNA that hybridizes to the endogenous KEAP1 gene in the patient, and a second nucleic acid sequence encoding a Cas nuclease, wherein the Cas nuclease cleaves the endogenous KEAP1 gene creating a KEAP1 knockout of the endogenous KEAP1 gene in the subject.

In some embodiments, the first and second nucleic acid sequences are on one vector. In some embodiments, the first and second nucleic acid sequences are on different vectors.

In some embodiments, the viral vectors are recombinant or rAAV. In some embodiments, the rAAV are AAV2 serotype. In some embodiments, the viral vectors are AAV8 serotype.

In some embodiments, the viral vectors are lentiviral vectors. In some embodiments, the lentiviral vectors are EAIV.

In some embodiments, the viral vectors comprise promoters which target particular cells. In some embodiments, these cells are retinal epithelial cells (RPE). In some embodiments, the targeted cells are retinal ganglion cells (RGC). In some embodiments, the promoter is one used specifically for interfering RNAs.

Any of the foregoing methods and compositions can be used to increase survival of cells, and to reprogram metabolism in cells. Such cells would include photoreceptor cells, neuronal cells, and retinal cells.

In some embodiments of any of the above aspects of the disclosure, the composition can further comprise a liposome, vesicle, synthetic vesicle, exosome, synthetic exosome, dendrimer, or nanoparticle.

Also provided herein are kits for practicing any of the disclosed methods.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

Pde6b^{H620QH620Q} abbreviated as Pde6b^{H620QH620Q} is a preclinical model of autosomal recessive RP.

FIG. 1 is a map of the design of the AAV8-VMD2 construct. FIG. 1A shows the control AAV8-VMD2-eGFP. FIG. 1B shows AAV8-VMD2-NRF2-eGFP construct.

FIG. 2 are graphs of ERGs of Pde6b^{H620QH620Q} preclinical recessive model after one month subretinal injection of AAV8-VMD2-PGC1α (solid line) and control non-injected eyes (dot lines). There is a statistically significant increase in amplitudes in scotopic a- (FIG. 2A), b- (FIG. 2B), and photopic b ((FIG. 2C) waves.

FIG. 3 is a map of the design of the AAV8::RPE65-Pgc1α-mcherry construct (FIG. 3A) and AAV8::RPE65-mcherry (control AAV) (FIG. 3B).

FIG. 4 is a boxplot of results from ERG from five Pde6b^{H620QH620Q} mice injected with AAV8::RPE65-Pgc1α-mcherry at P28. No mice were excluded because of cataract or retinal detachment. The serial intensities in scotopic a waves and b waves, and photopic b waves are compared. The white boxes represent the injected right eye, and empty boxes represent the noninjected control left eye. There are statistically significant differences between injected and noninjected eyes at some intensities in scotopic responses and all intensities in photopic responses. n=5; *P<0.05; **P<0.01; ***P<0.001

FIG. 5 is a boxplot of the results of ERG from five Pde6b^{H620QH620Q} mice (first litter) injected with AAV8::RPE65-Pgc1α-mcherry at P28 (maximum response combined rod and cone) and P47 (cone response). The a- and b-waves of maximum response and b-waves of cone response at 4 different time points and control AAV virus at P28 are in the first lane. There is a statistically significant difference between the injected and noninjected eyes at P28 and P47, but not at P55 and P72. The ERG results at the P55 and P72 were from mice that had been tested with ERG at either earlier time points. Some of these mice developed corneal opacity after the first ERG testing, affecting the ERG amplitude. *P<0.05, **P<0.01.

FIG. 6 show further results of one AAV8::RPE65-Pgc1α-mcherry treated pde6b^{H620QH620Q} mouse with functional and anatomical rescue. FIG. 6A is a graph of ERG response at P28 for AAV8::RPE65-Pgc1α-mcherry treated eye. FIG. 6B is a graph of ERG response at P28 for the control non-injected eye. The AAV8 treated eye exhibited higher amplitudes, indicating preserved photoreceptor function B. FIG. 6C shows histology at P64 of the AAV8::RPE65-Pgc1α-mcherry treated eye. FIG. 6D shows histology at P64 of the control eye. In FIGS. 6C and 6D, the left hand image shows the part of the eye from which tissue was taken. The center image shows the stained mid-periphery. The right hand image shows the optic nerve head. The histology results show a deceleration in photoreceptor degeneration in the injected eye of as compared to the control eye.

FIG. 7 shows less pigment migration of whole mount retinas from Pde6b^{H620Q/H620Q} eyes treated with AAV8::RPE65-Pgc1α-mcherry (FIG. 7A) and control non-injected eyes (FIG. 7B). Pigment migration is a marker of the severity of retinitis pigmentosa.

FIG. 8 shows images of whole mount retinas stained with cone-specific Arr3 antibody. FIG. 8A shows AAV8::RPE65-Pgc1α-mcherry treated Pde6b^{H620QH620Q} eyes and FIG. 8B shows untreated Pde6b^{H620Q/H620Q} controls.

FIG. 9 shows the map of the design of the AAV2/2-hSNCG constructs for PGC1═. FIG. 9A shows the design of the AAV2/2-hSNCG-PGC1α-eGFP. FIG. 9B shows the design of the AAV2/2-hSNCG-eGFP.

FIG. 10 shows the map of the design of the AAV2/2-hSNCG constructs NRF2. FIG. 10A shows the design of the AAV2/2-hSNCG-NRF2-eGFP. FIG. 10B shows the design of the AAV2/2-hSNCG-eGFP.

FIG. 11 shows the results of ablation of HIF on rod and cone cell survival and function. FIG. 11A is ERG data obtained at 4 and 6 weeks under dark- and light-adapted conditions to acquire scotopic, photopic, and mixed rod-cone b-wave amplitudes (μV). Traces of the retinal function of the Hif^{-/-}Pde6b^{H620Q/H620Q} (light trace) with Hif2a ablated in RPE were shown at 4 and 6 weeks compared to the age match Hif^{doxP/loxP}; Pde6b^{H620Q/H620Q} control (dark trace). FIG. 11B are graphs of the amplitudes of the ERG traces of the in Hif^{-/-}Pde6b^{H620Q/H620Q} (left hand bars) and plotted comparing to the age match control hif^{doxP/loxP};Pde6b^{H620Q/H620Q} (right hand bars). 4-5 mice were in each group. FIG. 11C are images of H & E staining of paraffin sections of retinae showing thicker ONL and OS layers and a greater width of photoreceptor outer nuclear (ONL) layer (top, yellow) and IS/OS layer (bottom, green) in Hif ^{-/-}; Pde6b^{H620Q/H620Q} compared with hif^{loxP/loxP}; Pde6b^{H620Q/H620Q} mice at 4 weeks. (Scale bar=50 μm). FIG. 11D are graphs of the width of ONL and IS/OS layer as measured and plotted.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

By “neuronal” is meant to refer to and include any cells which compose the central or peripheral nervous system.

By “retinal” is meant to refer to and include any light-sensitive cells in the eye as well the supporting cells that enable, facilitate, or are related to the phototransduction cascade.

The term “subject” as used in this application refers to animals in need of therapeutic or prophylactic treatment. Subjects include mammals, such as canines, felines, rodents, bovine, equines, porcines, ovines, and primates. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments, the “patient” is known or suspected of having a neurodegenerative disease or disorder including but not limited to glaucoma, retinitis pigmentosa (RP), age-related macular degeneration (AMD), autosomal dominant optic atrophy (ADOA), Alzheimer's disease, Parkinson's disease, Huntington's disease, Amyotrophic lateral sclerosis (ALS), and Lewy body dementia.

The phrase “therapeutically effective amount” is used herein to mean an amount sufficient to cause an improvement in a clinically significant condition in the subject, or delays or minimizes or mitigates one or more symptoms associated with the disease or disorder, or results in a desired beneficial change of physiology in the subject.

The terms “treat”, “treatment”, and the like refer to a means to slow down, relieve, ameliorate or alleviate at least one of the symptoms of the disease or disorder, or reverse the disease or disorder after its onset.

The terms “prevent”, “prevention”, and the like refer to acting prior to overt disease or disorder onset, to prevent the disease or disorder from developing or minimize the extent of the disease or disorder, or slow its course of development.

The term “cure” and the like means to heal, to make well, or to restore to good health or to allow a time without recurrence of disease so that the risk of recurrence is small

The term “in need thereof” would be a subject known or suspected of having or being at risk of having a neurodegenerative disease or disorder including but not limited to glaucoma, retinitis pigmentosa (RP), age-related macular degeneration (AMD), autosomal dominant optic atrophy (ADOA), Alzheimer's disease, Parkinson's disease, Huntington's disease, Amyotrophic lateral sclerosis (ALS), and Lewy body dementia.

The term “agent” as used herein means a substance that produces or is capable of producing an effect and would include, but is not limited to, chemicals, pharmaceuticals, biologics, small organic molecules, antibodies, nucleic acids, peptides, and proteins.

The term “composition” as used herein means a product which results from the mixing or combining of more than one element or ingredient.

As used herein, the term “carrier” refers to a diluent, adjuvant, excipient, or vehicle with which the therapeutic is administered, and includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art.

The term “pharmaceutically-acceptable” refers to molecular entities and compositions that do not produce an allergic or similar untoward reaction when administered to a host, such as gastric upset, dizziness and the like, when administered to a human, and approved by a regulatory agency of the Federal or a state government or listed in the U.S. Pharmacopeia or other generally recognized pharmacopeia for use in animals, and more particularly in humans

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector” includes any genetic element, such as a plasmid, phage, transposon, cosmid, chromosome, artificial chromosome, virus, or virion, which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene.

The term “expression vector” or “expression construct” or “construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA from a transcribed gene.

In some aspects, the disclosure provides isolated adeno-associated viral vectors (AAVs). As used herein with respect to AAVs, the term “isolated” refers to an AAV that has been isolated from its natural environment (e.g., from a host cell, tissue, or subject) or artificially produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities.

As used herein, the terms “AAV1,” “AAV2,” “AAV3,” “AAV4,” and the like refer to AAV vectors containing ITRs from AAV1, AAV2, AAV3, or AAV4, respectively, as well as capsid proteins from AAV1, AAV2, AAV3, or AAV4, respectively. The terms “AAV2/1,” “AAV2/8,” “AAV2/9,” and the like refer to pseudotyped AAV vectors containing ITRs from AAV2 and capsid proteins from AAV1, AAV8, or AAV9, respectively.

With respect to transfected host cells, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al., Virology 52:456 (1973), Sambrook et al., Molecular Cloning, a Laboratory Manual, Cold Spring Harbor Laboratories, New York (1989), Davis et al., Basic Methods in Molecular Biology, Elsevier (1986), and Chu et al., Gene 13:197 (1981). Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

Standard methods in molecular biology are described Sambrook, Fritsch and Maniatis Molecular Cloning, A Laboratory Manual, Cold Spring Harbor Laboratory Press, Cold Spring Harbor, N.Y. (1982 & 1989 2nd Edition, 2001 3rd Edition); Sambrook and Russell Molecular Cloning, 3rd ed., Cold Spring Harbor Laboratory Press, Cold Spring Harbor, N.Y. (2001); Wu Recombinant DNA, Vol. 217, Academic Press, San Diego, Calif.) (1993). Standard methods also appear in Ausbel, et al. Current Protocols in Molecular Biology, Vols.1-4, John Wiley and Sons, Inc. New York, N.Y. (2001).

### ABBREVIATIONS

RP—retinitis pigmentosa
ADOA—autosomal dominant optic atrophy
RPE—retinal pigment epithelium
RGC—retinal ganglion cell
OXPHOS—oxidative phosphorylation
ROS—reactive oxygen species
PGC1α—peroxisome proliferator-activated receptor gamma, coactivator 1 alpha
NRF2—nuclear factor erythroid 2-related factor
Keap1—Kelch-like ECH-associated protein 1
HIF—hypoxia-inducible factor
ERG—electroretinogram
PERG—patterned electroretinogram
ONL—outer nuclear layer

Disclosed herein is gene therapy for delaying the onset of and the prevention, treatment and/or cure of various neurodegenerative diseases causing blindness, including but not limited to retinitis pigmentosa (RP), glaucoma, macular degeneration and autosomal dominant optic atrophy (ADOA) as well as other neurodegenerative diseases including but not limited to Alzheimer's disease (AD), Parkinson's disease (PD), Huntington's disease, Amyotrophic lateral sclerosis (ALS), Lewy body dementia, and similar neurodegenerative conditions. While different mutations cause these diseases, the ultimate cause of these diseases is dysfunctional metabolism in the various cells involved in the diseases. Without being bound by any theory, by reprogramming the metabolism in these cells, these neurodegenerative diseases can be prevented, treated and/or cured regardless of the mutation causing the disease. Metabolism can be reprogrammed by targeted gene therapy that either increases or decreases particular factors and moreover delivers the therapy specifically to particular cells involved in various diseases.

Specifically disclosed herein are methods and compositions to delay the onset of, prevent, treat and/or cure a neurodegenerative disease by reprograming metabolism in retinal pigment epithelium (RPE). This is accomplished by:

Increasing the expression of peroxisome proliferator-activated receptor gamma, coactivator 1 alpha (PPARGC1A; also known as PGC1α);

Increasing the expression of nuclear factor erythroid 2-related factor (NRF2);

Decreasing or inhibiting the expression Kelch-like ECH-associated protein 1 (KEAP1); and/or

Decreasing or inhibiting the expression of hypoxia-inducible factor (HIF).

Also disclosed herein are methods and compositions to delay the onset of, prevent, treat and/or cure a neurodegenerative disease by reprograming metabolism in retinal ganglion cells (RGC). This is accomplished by:

Increasing the expression of peroxisome proliferator-activated receptor gamma, coactivator 1 alpha (PPARGC1A; also known as PGC1α); or

Increasing the expression of nuclear factor erythroid 2-related factor (NRF2); and/or

Decreasing or inhibiting the expression Kelch-like ECH-associated protein 1 (KEAP1).

These methods and compositions which target the gene delivery to the particular cells can be used to delay the onset of, prevent, treat and/or cure a variety of neurodegenerative diseases including but not limited to retinitis pigmentosa (RP), glaucoma, macular degeneration, autosomal dominant optic atrophy (ADOA), Alzheimer's disease, Parkinson's disease Huntington's disease, Amyotrophic lateral sclerosis (ALS), and Lewy body dementia. These methods and compositions can also be used to reprogram metabolism in cells, and increase survival of cells, including neuronal, retinal, and photoreceptor cells.

Glaucoma is associated with decreased mitochondrial biogenesis and increased oxidative stress due to mitochondrial dysfunction. Glaucoma affects more than 70 million people and is the most common neurodegenerative disease-causing blindness worldwide (Quigley and Broman 2006). It is, in fact, a group of multifactorial diseases, characterized by progressive RGC death and vision loss. A recent study in a mouse model of an inherited glaucoma demonstrated decreased mitochondrial biogenesis in the ganglion cell layer that was concurrent with the onset of optic nerve damage (Guo, et al 2014). Because mitochondrial biogenesis is the process that results in an increased number and/or volume of de novo mitochondria, increased mitochondrial biogenesis is postulated to be an adaptive measure to ensure that damaged mitochondria are replenished and a healthy population of mitochondria can be maintained. Furthermore, accumulating evidence links oxidative stress to mitochondrial dysfunction in glaucomatous neurodegeneration. Abnormal mitochondrial metabolism and impaired oxidative phosphorylation (OXPHOS) have been shown to lead to mitochondrial damage and oxidative stress due to increased ROS levels that subsequently result in RGC apoptosis. No prior studies have assessed the role of mitochondrial biogenesis and the antioxidant response in promoting RGC survival in glaucoma. Current clinical approaches for glaucoma and other optic atrophies are focused on lowering intraocular pressure, specifically inhibition of RGC death; however, reducing the intraocular pressure does not reduce the glaucomatous visual field loss in roughly one-half of patients. Thus, there is a great need for mitochondria-targeted therapies for diseases associated with RGC death.

Autosomal dominant optic atrophy (ADOA) is associated with mitochondrial dysfunction. ADOA is the most common hereditary optic neuropathy, with an estimated disease prevalence of 1:12,000 to 1:50,000.17 It is associated with mutations in the nuclear OPA1 gene. OPA1 encodes the OPA1 protein, a mitochondrial dynamin-related GTPase that controls mitochondrial dynamics (inner membrane fusion), cristal integrity, energetics, and mitochondrial DNA (mtDNA) maintenance (Delettre, et al. 2002). Prior studies on OPA1 function have shown that OPA1 deficiency causes defects in cristae organization, biogenesis, oxidative phosphorylation (OXPHOS), and maintenance of the mitochondrial membrane potential, increases apoptotic sensitivity, and decreases mtDNA levels/stability and energetic activity. However, how mitochondrial dysfunction causes RGC death has not been clearly delineated.

Retinal ganglion cell (RGC) death is associated with mitochondrial dysfunction. RGCs are exquisitely vulnerable to mitochondrial dysfunction due to the highly compartmentalized energy demands associated with their unique cytoarchitecture, with long axons that extend through the optic nerve to form distal terminals and connections in the brain (Wang, et al. 2003). Mitochondrial biogenesis is a critical component of mitochondrial quality control to ensure replacement of damaged mitochondria, and reductions in mitochondrial biogenesis are associated with many neurodegenerative disorders, such as glaucoma and optic atrophy (Guo, et al. 2014; MacVivar and Langer, 2016). Accumulating evidence links oxidative stress to mitochondrial dysfunction in RGC death, and abnormal mitochondrial metabolism and impaired oxidative phosphorylation (OXPHOS) lead to mitochondrial damage and oxidative stress due to increased levels of reactive oxygen species (ROS), resulting in RGC apoptosis. How mitochondrial dysfunction leads to RGC death as well as the mechanism(s) required for maintaining healthy mitochondrial populations in RGCs are not fully understood. With the use of a novel ADOA mouse model which shows reduced RGC numbers and function, and other mouse models of RGC death, shown herein is that restoring mitochondrial function prevents RGC death, which in turn can be used to treat, prevent and/or cure ADOA, glaucoma and other neurodegenerative diseases.

RP is the most common inherited retinal dystrophy with a worldwide prevalence of about 1 in 3,000 and a total of greater than 1 million affected individuals. RP is caused by any one of greater than 71 mutations that primarily cause rod photoreceptor death. Although cones are not directly killed by these mutations, their death always follows rod death, regardless of the specific mutation, usually after the major rod death phase (Campochiaro, et al. 2018; Punzo, et al. 2009). Because cones are essential for daylight, color, and central vision, it is their death that causes blindness. Previous studies and clinical trials for RP have focused on augmenting or repairing a single gene; however, these strategies, even if successful, are applicable to only to patients carrying a single specific mutation (Takahashi, et al. 2018). The FDA recently approved two potential therapies for RP: a retinal prosthesis, approved only for patients with end-stage RP and RPE65 gene therapy, approved only for patients carrying the RPE65 mutation (Duncan, et al. 2018). There is a great need for developing therapies targeting a pathway common to cone death in RP that could serve as a more widely applicable treatment for RP resulting from numerous rod-specific mutations.

Increasing evidence suggests that overt oxidative stress, which represents an imbalance between the production and elimination of reactive oxygen species (ROS), within the retina plays an important role in the progression of cone death in RP. Because rods comprised 95% of the cells in the outer nuclear layer and consume the majority of oxygen delivered to the outer retina, their death in RP results in high levels of oxygen that cause excessive tissue oxygen and increase the production of superoxide radicals. The excess superoxide radicals generate other ROS, for example peroxynitrite, which attacks lipids, proteins and DNA, and subsequently cone death. Prior studies demonstrated cone-preserving effects in animal models of RP with systemic administration of antioxidants, suggesting oxidative stress play an important role of cone death in RP (Lee, et al 2011).

The Nrf2/KEAP1 (Kelch-like ECH-associated protein 1) signaling pathway is one of the most important cell antioxidant defense and survival pathways. Several studies demonstrated that NRF2 has protective role against oxidative stress in several eye diseases, (Xiong, et al. 2015; Larabee, et al. 2016). However, there is a knowledge gap in which cells in the outer retina contribute the major ROS that attribute to cone death in RP, and therefore, providing little guidance for targeted treatment. No prior studies have assessed whether enhancing antioxidant response in specific cells in the retina can promote photoreceptor survival. The lack of understanding which cells in the outer retina contribute the major ROS presents a critical knowledge gap. Shown herein is that enhancing cell-specific antioxidant response can aid in preventing photoreceptor death which again in turn, can prevent, treat and/or cure disease.

A further gene therapy solution to RP centers around restoring the balance of two universal metabolic processes and the “metabolic coupling” hypothesis: oxidative phosphorylation (OXPHOS) which turns glucose to energy and anabolism which turns glucose to fat in retinal pigment epithelium (RPE) and photoreceptors (rods (95%) and cones (5%)). These two processes are intimately connected. In healthy eyes, rod photoreceptors take up glucose from the RPE and convert it into fat and lactate. The RPE, in turn, consumes the generated lactate and produces energy via OXPHOS, an ATP-generating pathway that suppresses anabolism. When rod cells start to die or age, the concentration of lactate decrease, prompting the RPE to use glucose to produce fats and lipids. When the RPE has consumed nearly all of the available glucose, the cone cells will also die. See Zhang, et al. 2016A and Zhang, et al. 2016B. Thus, the central hypothesis is that reprogramming RPE metabolism can promote cone survival in RP independently of the underlying specific gene mutations.

Defining the mechanisms underlying cone death in RP is challenging, as greater than 95% of mammalian photoreceptors are rods, and this metabolic coupling hypothesis is based on studies focused on rod-RPE metabolic coupling. Thus, little is known about cellular metabolism in cones or metabolic coupling between cones and RPE. Given that cone death leads to blindness in RP, a better understanding of the role of cones in the retinal metabolic coupling is crucial for developing therapies for RP.

The metabolic reprogramming to treat disease can be accomplished by upregulating or downregulating certain transcription factors in RGC, RPE and photoreceptor cells. The upregulation or downregulation of these transcription factors in the specific cells will be accomplished using cell specific promoter which will deliver the various agents to the specific cells.

One promising interventional approach is to restore the metabolic mutualism between RPE and photoreceptors by modulating nodes for metabolic compartmentation in a cell-specific manner Two genes that have received attention as attractive therapeutic targets are hypoxia-inducible factor (HIF) and PPar(gamma)-coactivator-1 alpha (PGC1α), both of which encode for transcription factors that play critical roles in oxidative phosphorylation. It is known that HIF and PGC1α are negative and positive regulators of oxidative phosphorylation, respectively. To evaluate the rescue effects of autonomously reprogramming RPE cells from a pathological-glycolytic to neuroprotective-oxidative state, RPE-specific ablation of HIF and overexpression of PGC1α in retinitis pigmentosa (RP) mouse models were separately induced. Notably, treated mice exhibited preserved photoreceptor morphology and function, validating the therapeutic efficacy of this innovative imprecision medicine approach. Given that metabolic disequilibrium underlies a vast array of neurodegenerative diseases from age-related macular degeneration to Parkinson's disease, the clinical impact and significance of our therapeutic strategy cannot be overstated.

Peroxisome proliferator-activated receptor gamma, coactivator 1 alpha (PPARGC1A; also known as PGC1α) is thought to induce mitochondrial biogenesis, including synthesis of mtDNA, proteins, and membranes. The PGC1α coactivator is a master regulator of mitochondrial biogenesis (Fernandez-Marcus, et al. 2011). PGC-1α remodels cell metabolism from glycolysis to OXPHOS by regulating mitochondrial activity and suppressing glycolysis.

In the brain, PGC1α knockdown decreased the number of mitochondria in hippocampal dendrites, resulting in lower density of dendritic spines and synapses, whereas PGC1α overexpression boosted mitochondrial biogenesis and synaptogenesis. The role of PGC1α in the eye is controversial and has been little studied in vivo.

Support for the beneficial effects of downregulating or ablating HIF is that in aging, HIF expression is increased two times in RPE (Wang, et al. 2020). Thus, downregulation of Hif1a/2a in RPE using targeted delivery would reprogram metabolism to a healthy state, i.e., increase of OXPHOS in in RPE and increase of anabolism in photoreceptor cells.

Nuclear factor erythroid 2-related factor (NRF2) is a key nuclear transcription factor for the systemic antioxidant defense system. Under normal physiological conditions, NRF2 turns over rapidly and is found at low levels due to constant degradation by the ubiquitin-proteasome system (McMahon, et al. 2004). Under stress conditions, NRF2 dissociates from Kelch-like ECH-associated protein 1 (KEAP1, a primary Nrf2 inhibitor) and is translocated into the nucleus, where it binds to antioxidant response elements (AREs) in DNA promoter regions. NRF2 then initiates transcription of ARE-related genes (NQO1, HO-1, GCLC, GCLM, and glutathione synthetase), all of which detoxify ROS through regulation of glutathione. Several studies have demonstrated that Nrf2 protects against oxidative stress in diabetic retinopathy and retinal ischemia-reperfusion, and promotes neuronal survival in neurodegeneration and optic neuritis (Larabee, et al. 2016; Xiong, et al. 2015). However, little is known about whether induction of the Nrf2/Keap1/ARE signaling pathway can be targeted to promote neuroprotection of RGCs.

A recent study in a mouse model of RP reported a detrimental effect of retinal PGC1α overexpression (Xiong, et al. 2015). A potential issue with this in vivo mouse study is that it used the human CMV promoter. Although this promoter drives robust expression in cones, some expression in rods and RPE were also noted by the authors; thus the use of this promoter led to nonspecific overexpression. Therefore, further studies using an RPE-specific promoter or Cre-driver could be helpful in determining the role of PGC1α in RPE biology. Shown herein is the use of cell-specific promoters producing a beneficial effect of overexpression of both PGC1α and NRF2 as well as the inhibition of HIF.

Shown herein is gene therapy which rescues metabolic imbalance thus slowing progression of disease that is not mutation specific. The increase or overexpression of PGC1α and NRF2 and inhibition or ablation of HIF and KEAP1 rescues metabolic imbalance in particular cells leading to the treatment, prevention and curing of disease. Using cell-specific promoters in the disclosed gene therapy ensures the nucleic acids are delivered in a targeted fashion. Because this technology targets a disease mechanism rather than a single gene, it has the potential to treat all RP patients. Additionally, this therapeutic approach can be applied to other degenerative conditions that involve metabolic dysregulation, such as Alzheimer's disease.

### Methods and Compositions to Overexpress or Increase PGC1α and NRF2

The compositions and methods of the present disclosure may be used to delay the onset of, treat, prevent, and/or cure a variety of pathologies. For example, gene therapy methods described herein may involve administration of one or more compositions containing a nucleic acid encoding a protein of which overexpression can treat, prevent and/or cure a disease, in particular a neurodegenerative disease, and more particular a neurodegenerative disease causing blindness, including but not limited to glaucoma, retinitis pigmentosa (RP), age-related macular degeneration (AMD), and autosomal dominant optic atrophy (ADOA). In some embodiments the methods and compositions disclosed herein can be used to prevent, treat and/or cure Alzheimer's disease. In some embodiments the methods and compositions disclosed herein can be used to prevent, treat and/or cure other neurodegenerative diseases including but not limited to Parkinson's disease Huntington's disease, Amyotrophic lateral sclerosis (ALS), and Lewy body dementia.

In some embodiments, the protein to be overexpressed is PGC1α. In some embodiments, the protein to be overexpressed is NRF2.

In some embodiments, the present disclosure provides methods of delaying the onset of, treating, preventing, curing, and/or reducing the severity or extent of a neurodegenerative disease or disorder, by administering to a subject in need thereof a therapeutically effective amount of a composition, or compositions, such as a viral vector (e.g., an AAV), comprising a nucleic acid encoding PGC1α and/or NRF2. In some embodiments, the viral vector is an AAV, such as rAAV2, AAV10, AAV2/10, AAV9 or an AAV2/9. Additionally, the compositions, e.g., viral vectors, comprise cell specific promoters to target the delivery of the nucleic acids to the proper cells. In some embodiments, the cells to be targeted are retinal ganglion cells and the promoter used includes but is not limited to hSNCG, and Ple345 (NEFL). In some embodiments, the cells to be targeted are retinal pigment epithelium cells and the promoter used includes but is not limited to VMD2, RPE65 and TYR.

Any suitable viral system could be utilized for increasing decreasing PGC1α and/or NRF2 including AAV, lentiviral vectors, or other suitable vectors.

In some embodiments, the composition or compositions (e.g., viral vector, such as an AAV) comprising a nucleic acid encoding PGC1α and/or NRF2 is administered as soon as neurodegenerative disease or disorder is diagnosed or suspected. These compositions may be administered alone or in combination with other agents for the treatment of neurodegenerative diseases or disorders.

Nucleic acid sequences of transgenes described herein may be designed based on the knowledge of the specific composition (e.g., viral vector) that will express the transgene. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

In some embodiments of the current disclosure the transgenes would encode a functional protein including but not limited to PGC1α and NRF2.

By “PGC1α”, “PGC1α”, Pgc1α″, and “Pgc1α” is meant to include the DNA, RNA, mRNA, cDNA, recombinant DNA or RNA, or the protein arising from the PGC1α.

It is noted that as used herein PGC1α can refer to the gene or the protein encoded for by the gene, as appropriate in the specific context utilized. Additionally, in certain contexts, the reference will be to the mouse gene or protein, and in others the human gene or protein as appropriate in the specific context.

The human PGC1α gene (GenBank: 10891) can be used to obtain a transgene.

In some embodiments, the transgene encodes PGC1α. The PGC1α may have an amino acid sequence that is at least 85% identical to the amino acid sequence of human PGC1α (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of human PGC1α). In some embodiments, the PGC1α has an amino acid sequence that is at least 90% identical to the amino acid sequence of human PGC1α (e.g., an amino acid sequence that is 90%, 91%, 92%, 93%, 94%, 95%, 96%, 97%, 98%, 99%, or 100% identical to the amino acid sequence of human PGC1α). In some embodiments, the PGC1α has an amino acid sequence that is at least 95% identical to the amino acid sequence of human PGC1α (e.g., an amino acid sequence that is 95%, 96%, 97%, 98%, 99%, or 100% identical to the amino acid sequence of human PGC1α).

In some embodiments, the PGC1α has an amino acid sequence that differs from human PGC1α by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the PGC1α has an amino acid sequence that differs from human PGC1α by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding PGC1α has a nucleic acid sequence that is at least 70% identical to the nucleic acid sequence of human PGC1α (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human PGC1α). In some embodiments, the transgene encoding PGC1α has a nucleic acid sequence that is at least 85% identical to the nucleic acid sequence of human PGC1α (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human PGC1α). In some embodiments, the transgene encoding PGC1α has a nucleic acid sequence that is at least 90% identical to the nucleic acid sequence of human PGC1α (e.g., a nucleic acid sequence that is 90%, 91%, 92%, 93%, 94%, 95%, 96%, 97%, 98%, 99%, or 100% identical to the nucleic acid sequence of human PGC1α). In some embodiments, the transgene encoding PGC1α has a nucleic acid sequence that is at least 95% identical to the nucleic acid sequence of human PGC1α (e.g., a nucleic acid sequence that is 95%, 96%, 97%, 98%, 99%, or 100% identical to the nucleic acid sequence of human PGC1α).

In some embodiments, the transgene encodes PGC1α comprising SEQ ID NO: 1. The PGC1α may have an amino acid sequence that is at least 85% identical to the amino acid sequence of SEQ ID NO: 1 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 1). In some embodiments, the PGC1α has an amino acid sequence that is at least 90% identical to the amino acid sequence of SEQ ID NO: 1 (e.g., an amino acid sequence that is 90%, 91%, 92%, 93%, 94%, 95%, 96%, 97%, 98%, 99%, or 100% identical to the amino acid sequence of SEQ ID NO:1). In some embodiments, the PGC1α has an amino acid sequence that is at least 95% identical to the amino acid sequence of SEQ ID NO: 1 (e.g., an amino acid sequence that is 95%, 96%, 97%, 98%, 99%, or 100% identical to the amino acid sequence of SEQ ID NO:1).

In some embodiments, the PGC1α has an amino acid sequence that differs from SEQ ID NO: 1 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the PGC 1α has an amino acid sequence that differs from SEQ ID NO: 1 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding PGC1α has a nucleic acid sequence that is at least 70% identical to the nucleic acid sequence encoding SEQ ID NO: 1 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 1). In some embodiments, the transgene encoding PGC1α has a nucleic acid sequence that is at least 85% identical to the nucleic acid sequence encoding SEQ ID NO: 1 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 1). In some embodiments, the transgene encoding PGC1α has a nucleic acid sequence that is at least 90% identical to the nucleic acid sequence encoding SEQ ID NO: 1 (e.g., a nucleic acid sequence that is 90%, 91%, 92%, 93%, 94%, 95%, 96%, 97%, 98%, 99%, or 100% identical to the nucleic acid sequence encoding SEQ ID NO: 1). In some embodiments, the transgene encoding PGC1α has a nucleic acid sequence that is at least 95% identical to the nucleic acid sequence encoding SEQ ID NO: 1 (e.g., a nucleic acid sequence that is 95%, 96%, 97%, 98%, 99%, or 100% identical to the nucleic acid sequence encoding SEQ ID NO: 1).

In some embodiments, the transgene encoding PGC1α is codon optimized to increase efficiency.

Codon optimization tools are known in the art.

By “NRF2”, “NRF2”, “Nrf2”, and “Nrf2” is meant to include the DNA, RNA, mRNA, cDNA, recombinant DNA or RNA, or the protein arising from the NRF2.

It is noted that as used herein NRF2 can refer to the gene or the protein encoded for by the gene, as appropriate in the specific context utilized. Additionally, in certain contexts, the reference will be to the mouse gene or protein, and in others the human gene or protein as appropriate in the specific context.

The human NRF2 gene (GenBank: 4780) can be used to obtain a transgene.

In some embodiments, the transgene encodes NRF2. The NRF2 may have an amino acid sequence that is at least 85% identical to the amino acid sequence of human NRF2 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of human NRF2). In some embodiments, the NRF2 has an amino acid sequence that is at least 90% identical to the amino acid sequence of human NRF2 (e.g., an amino acid sequence that is 90%, 91%, 92%, 93%, 94%, 95%, 96%, 97%, 98%, 99%, or 100% identical to the amino acid sequence of human NRF2). In some embodiments, the NRF2 has an amino acid sequence that is at least 95% identical to the amino acid sequence of human NRF2 (e.g., an amino acid sequence that is 95%, 96%, 97%, 98%, 99%, or 100% identical to the amino acid sequence of human NRF2).

In some embodiments, the NRF2 has an amino acid sequence that differs from human NRF2 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the NRF2 has an amino acid sequence that differs from human NRF2 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding NRF2 has a nucleic acid sequence that is at least 70% identical to the nucleic acid sequence of human NRF2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human NRF2). In some embodiments, the transgene encoding NRF2 has a nucleic acid sequence that is at least 85% identical to the nucleic acid sequence of human NRF2 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human NRF2). In some embodiments, the transgene encoding NRF2 has a nucleic acid sequence that is at least 90% identical to the nucleic acid sequence of human NRF2 (e.g., a nucleic acid sequence that is 90%, 91%, 92%, 93%, 94%, 95%, 96%, 97%, 98%, 99%, or 100% identical to the nucleic acid sequence of human NRF2). In some embodiments, the transgene encoding NRF2 has a nucleic acid sequence that is at least 95% identical to the nucleic acid sequence of human NRF2 (e.g., a nucleic acid sequence that is 95%, 96%, 97%, 98%, 99%, or 100% identical to the nucleic acid sequence of human NRF2).

In some embodiments, the transgene encodes NRF2 comprising SEQ ID NO: 2. The NRF2 may have an amino acid sequence that is at least 85% identical to the amino acid sequence of SEQ ID NO: 2 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 2). In some embodiments, the NRF2 has an amino acid sequence that is at least 90% identical to the amino acid sequence of SEQ ID NO: 2 (e.g., an amino acid sequence that is 90%, 91%, 92%, 93%, 94%, 95%, 96%, 97%, 98%, 99%, or 100% identical to the amino acid sequence of SEQ ID NO: 2). In some embodiments, the NRF2 has an amino acid sequence that is at least 95% identical to the amino acid sequence of SEQ ID NO: 2 (e.g., an amino acid sequence that is 95%, 96%, 97%, 98%, 99%, or 100% identical to the amino acid sequence of SEQ ID NO: 2).

In some embodiments, the NRF2 has an amino acid sequence that differs from SEQ ID NO: 2 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the NRF2 has an amino acid sequence that differs from SEQ ID NO: 2 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding NRF2 has a nucleic acid sequence that is at least 70% identical to the nucleic acid sequence encoding SEQ ID NO: 2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 2). In some embodiments, the transgene encoding NRF2 has a nucleic acid sequence that is at least 85% identical to the nucleic acid sequence encoding SEQ ID NO: 2 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence encoding SEQ ID NO: 2). In some embodiments, the transgene encoding NRF2 has a nucleic acid sequence that is at least 90% identical to the nucleic acid sequence encoding SEQ ID NO: 2 (e.g., a nucleic acid sequence that is 90%, 91%, 92%, 93%, 94%, 95%, 96%, 97%, 98%, 99%, or 100% identical to the nucleic acid sequence encoding SEQ ID NO: 2). In some embodiments, the transgene encoding NRF2 has a nucleic acid sequence that is at least 95% identical to the nucleic acid sequence encoding SEQ ID NO: 2 (e.g., a nucleic acid sequence that is 95%, 96%, 97%, 98%, 99%, or 100% identical to the nucleic acid sequence encoding SEQ ID NO: 2).

In some embodiments, the transgene encoding NRF2 is codon optimized to increase efficiency.

Codon optimization tools are known in the art.

### Methods and Compositions to Inhibit, Ablate or Decrease HIF and KEAP1

The compositions and methods of the present disclosure may be used to delay the onset of, treat, prevent, and/or cure a variety of pathologies. For example, gene therapy methods described herein may involve administration of one or more compositions containing an inhibitor of a nucleic acid or protein can treat, prevent and/or cure a disease, in particular a neurodegenerative disease, and more particular a neurodegenerative disease-causing blindness, including but not limited to glaucoma, retinitis pigmentosa (RP), age-related macular degeneration (AMD), and autosomal dominant optic atrophy (ADOA). In some embodiments, the methods and compositions disclosed herein can be used to prevent, treat and/or cure Alzheimer's disease. In some embodiments, the methods and compositions disclosed herein can be used to prevent, treat and/or cure other neurodegenerative diseases including but not limited to Parkinson's disease Huntington's disease, Amyotrophic lateral sclerosis (ALS), and Lewy body dementia.

In some embodiments, the protein to be inhibited is HIF.

By “HIF,” “HIF,” “Hif,” “Hif” is meant to include the DNA, RNA, mRNA, cDNA, recombinant DNA or RNA, or the protein arising from the HIF gene or HIF interactors. Moreover, HIF as used herein is meant to include HIF1A, HIF2A, and HIF3A. The human reference sequences for HIF1A and HIF2A can be found at GenBank 3091 and 2034, respectively. The sequences for HIF3A can be found at GenBank Accession Nos. NM-022462, NM-152794 and NM-152795.

It is noted that as used herein HIF can refer to the gene or the protein encoded for by the gene, as appropriate in the specific context utilized. Additionally, in certain contexts, the reference will be to the mouse gene or protein, and in others the human gene or protein as appropriate in the specific context.

Any isoform of any HIF may be inhibited by the present inhibitors. The present inhibitors may target the wild-type or mutant form of HIF.

In some embodiments, the protein to be inhibited is KEAP1.

By “KEAP1,” “KEAP1,” “Keap1,” “Keap1” is meant to include the DNA, RNA, mRNA, cDNA, recombinant DNA or RNA, or the protein arising from the KEAP1 gene or KEAP1 interactors. The human reference sequence can be found at GenBank 9817.

It is noted that as used herein KEAP1 can refer to the gene or the protein encoded for by the gene, as appropriate in the specific context utilized. Additionally, in certain contexts, the reference will be to the mouse gene or protein, and in others the human gene or protein as appropriate in the specific context.

Any isoform of any KEAP1 may be inhibited by the present inhibitors. The present inhibitors may target the wild-type or mutant form of KEAP1.

As used herein, the term “inhibitor” refers to agents capable of down-regulating or otherwise decreasing or suppressing the amount/level and/or activity of HIF and/or KEAP1.

The mechanism of inhibition may be at the genetic level (e.g., interference with or inhibit expression, transcription or translation, etc.) or at the protein level (e.g., binding, competition, etc.).

A wide variety of suitable inhibitors may be employed, guided by art-recognized criteria such as efficacy, toxicity, stability, specificity, and half-life.

In some embodiments, the present disclosure provides methods of treating, preventing, curing, and/or reducing the severity or extent of a neurodegenerative disease or disorder, by administering to a subject in need thereof a therapeutically effective amount of a composition, or compositions, such as a viral vector (e.g., an AAV or lentiviral vector), comprising an inhibitor of HIF or KEAP1. In some embodiments, the viral vector is an AAV, such as rAAV2, AAV10, AAV2/10, AAV9 or an AAV2/9.

Any suitable viral knockdown system could be utilized for decreasing HIF or KEAP1 mRNA levels—including AAV, lentiviral vectors, or other suitable vectors.

In some embodiment, the vector is a lentivirus. In some embodiments, the lentiviral vector is HIV based. In some embodiments, the lentiviral vector is EIAV based.

Additionally, the compositions, e.g., viral vectors, comprise cell specific promoters to target the delivery of the nucleic acids to the proper cells. In some embodiments, the cells to be targeted are retinal pigment epithelium cells and the promoter used includes but is not limited to VMD2, RPE65 and TYR. In some embodiments, the cells to be targeted are retinal ganglion cells and the promoter used includes but is not limited to hSNCG, and Ple345 (NEFL).

Additionally, specifically targeted delivery of HIF or KEAP1 blocking molecule (nucleic acid, peptide, or small molecule) could be delivered by targeted liposome, nanoparticle or other suitable means.

In some embodiments, the composition or compositions (e.g., viral vector, such as an AAV) comprising the inhibitor of HIF or KEAP1 is administered as soon as neurodegenerative disease or disorder is diagnosed or suspected. These compositions may be administered alone or in combination with other agents for the treatment of neurodegenerative diseases or disorders.

Endonucleases

Methods for modification of genomic DNA are well known in the art. For example, methods may use a DNA digesting agent to modify the DNA by either the non-homologous end joining DNA repair pathway (NHEJ) or the homology directed repair (HDR) pathway. The term “DNA digesting agent” refers to an agent that is capable of cleaving bonds (i.e., phosphodiester bonds) between the nucleotide subunits of nucleic acids.

In one embodiment, the DNA digesting agent is a nuclease. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyses the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences.

HIF or KEAP1 may be inhibited by using a sequence-specific endonuclease that target the gene encoding HIF or KEAP1.

Non-limiting examples of the endonucleases include a zinc finger nuclease (ZFN), a ZFN dimer, a ZFNickase, a transcription activator-like effector nuclease (TALEN), or a RNA-guided DNA endonuclease (e.g., CRISPR/Cas). Meganucleases are endonucleases characterized by their capacity to recognize and cut large DNA sequences (12 base pairs or greater). Any suitable meganuclease may be used in the present methods to create double-strand breaks in the host genome, including endonucleases in the LAGLIDADG and PI-Sce family.

One aspect of the present disclosure provides RNA-guided endonucleases. RNA-guided endonucleases also comprise at least one nuclease domain and at least one domain that interacts with a guide RNA. An RNA-guided endonuclease is directed to a specific nucleic acid sequence (or target site) by a guide RNA. The guide RNA interacts with the RNA-guided endonuclease as well as the target site such that, once directed to the target site, the RNA-guided endonuclease is able to introduce a double-stranded break into the target site nucleic acid sequence. Since the guide RNA provides the specificity for the targeted cleavage, the endonuclease of the RNA-guided endonuclease is universal and can be used with different guide RNAs to cleave different target nucleic acid sequences.

One example of a RNA guided sequence-specific nuclease system that can be used with the methods and compositions described herein includes the CRISPR system (Wiedenheft, et al. 2012 Nature 482:331-338; Jinek, et al. 2012 Science 337:816-821; Mali, et al. 2013 Science 339:823-826; Cong, et al. 2013. Science 339:819-823). The CRISPR (Clustered Regularly Interspaced Short Palindromic Repeats) system exploits RNA-guided DNA-binding and sequence-specific cleavage of target DNA. The guide RNA/Cas combination confers site specificity to the nuclease. A single guide RNA (sgRNA) contains about 20 nucleotides that are complementary to a target genomic DNA sequence upstream of a genomic PAM (protospacer adjacent motifs) site (e.g., NGG) and a constant RNA scaffold region. The Cas (CRISPR-associated) protein binds to the sgRNA and the target DNA to which the sgRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong, et al. 2013 Science 339:819-823). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein.

In one embodiment, the methods of the present disclosure comprise using one or more sgRNAs to target and/or remove or suppress HIF.

In some embodiments, the sgRNAs that target HIF and/or to remove or suppress HIF are set forth in Tables 1 and 2.

TABLE 1
sgRNAs for use to remove or suppress HIF

| PAM | SEQUENCE | TARGET GENE |
|---|---|---|
| TGG | GTTATGGTTCTCACAGATGA | HIF1A EXON 3-4 |
| | (SEQ ID NO: 3) | |
| AGG | TACTCATCCATGTGACCATG | HIF1A EXON 3-4 |
| | (SEQ ID NO: 4) | |
| GGG | AGATGGGAGCTCACACTGTG | HIF2A EXON 2 |
| | (SEQ D NO: 5) | |
| GGG | CAGCATGTTCCTATATGCAG | HIF2A EXON 2 |
| | (SEQ ID NO: 6) | |

TABLE 2
Additional sgRNAs Targeting HIF

| | | SPECIFICITY | EFFICIENCY |
|---|---|---|---|
| PAM | SEQUENCE | SCORE | SCORE |
| AGG | CTTGTACCTGAAAGCCTTGG | 38.0998779 | 48.3847139 |
| | (SEQ ID NO: 7) | | |
| AGG | AAGTGCACGGTCACCAACAG | 41.1576188 | 64.0211178 |
| | (SEQ ID NO: 8) | | |
| TGG | ATGTGGGATGGGTGCTGGAT | 37.365837 | 30.548208 |
| | (SEQ ID NO: 9) | | |
| GGG | GGGGGATGTCCATGTGGGAT | 38.9185111 | 43.4462625 |
| | (SEQ ID NO: 10) | | |
| AGG | GATGGCCTTGCCATAGGCTG | 40.8643978 | 52.2189843 |
| | (SEQ ID NO: 11) | | |
| AGG | ACTGACCAGATATGACTGTG | 38.6271508 | 76.093746 |
| | (SEQ ID NO: 12) | | |
| TGG | AAGGCCACTGCTTGGTGACC | 37.8177689 | 46.5173893 |
| | (SEQ ID NO: 13) | | |

In one embodiment, the methods of the present disclosure comprise using one or more sgRNAs to target and/or remove or suppress KEAP1.

In some embodiments, the sgRNAs to target and/or remove or suppress KEAP1 target KEAP1 Exon 1. In some embodiments, the sgRNA to target and/or remove or suppress KEAP1 has one of the following sequences:

(SEQ ID NO: 14)
AGCGTCCTGCCATTGGCTTG;
(SEQ ID NO: 15)
GCGGGAGCAGGGCATGGAGG;
(SEQ ID NO: 16)
CCGACAGTCGCTCAGCTACC;
(SEQ ID NO: 17)
ACAGTCGCTCAGCTACCTGG;
(SEQ ID NO: 18)
GAGGACACACTTCTCGCCCA;
(SEQ ID NO: 19)
GACCAGGTAGTCCTTGCAGC;
AND
(SEQ ID NO: 20)
CCAGGTAGCTGAGCGACTGT.

In one embodiment, the DNA digesting agent can be a site-specific nuclease. In another embodiment, the site-specific nuclease may be a Cas-family nuclease. In a more specific embodiment, the Cas nuclease may be a Cas9 nuclease.

In one embodiment, Cas protein may be a functional derivative of a naturally occurring Cas protein.

In some embodiments, the nucleotide sequence encoding the Cas (e.g., Cas9) nuclease is modified to alter the activity of the protein. In some embodiments, the Cas (e.g., Cas9) nuclease is a catalytically inactive Cas (e.g., Cas9) (or a catalytically deactivated/defective Cas9 or dCas9). In one embodiment, dCas (e.g., dCas9) is a Cas protein (e.g., Cas9) that lacks endonuclease activity due to point mutations at one or both endonuclease catalytic sites (RuvC and HNH) of wild type Cas (e.g., Cas9). For example, dCas9 contains mutations of catalytically active residues (D10 and H840) and does not have nuclease activity. In some cases, the dCas has a reduced ability to cleave both the complementary and the non-complementary strands of the target DNA. In some cases, the dCas9 harbors both D10A and H840A mutations of the amino acid sequence of S. pyogenes Cas9. In some embodiments when a dCas9 has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the Cas protein can still bind to target DNA in a site-specific manner, because it is still guided to a target polynucleotide sequence by a DNA-targeting sequence of the subject polynucleotide (e.g., gRNA), as long as it retains the ability to interact with the Cas-binding sequence of the subject polynucleotide (e.g., gRNA).

The present methods and systems may use CRISPR deletion (CRISPRd). CRISPRd capitalizes on the tendency of DNA repair strategies to default towards NHEJ and does not require a donor template to repair the cleaved strand. Instead, Cas creates a DSB in the gene harboring a mutation first, then NHEJ occurs, and insertions and/or deletions (INDELs) are introduced that corrupt the sequence, thus either preventing the gene from being expressed or proper protein folding from occurring. This strategy may be particularly applicable for dominant conditions, in which case knocking out the mutated, dominant allele and leaving the wild type allele intact may be sufficient to restore the phenotype to wild type.

In addition to well characterized CRISPR-Cas system, a new CRISPR enzyme, called Cpf1 (Cas protein 1 of PreFran subtype) may be used in the present methods and systems (Zetsche et al. 2015. Cell). Cpf1 is a single RNA-guided endonuclease that lacks tracrRNA, and utilizes a T-rich protospacer-adjacent motif. The authors demonstrated that Cpf1 mediates strong DNA interference with characteristics distinct from those of Cas9. Thus, in one embodiment of the present invention, CRISPR-Cpf1 system can be used to cleave a desired region within the targeted gene.

In further embodiment, the nuclease is a transcription activator-like effector nuclease (TALEN). TALENs contains a TAL effector domain that binds to a specific nucleotide sequence and an endonuclease domain that catalyzes a double strand break at the target site (PCT Patent Publication No. WO2011072246; Miller et al., 2011 Nat. Biotechnol. 29:143-148; Cermak et al., 2011 Nucleic Acid Res. 39:e82). Sequence-specific endonucleases may be modular in nature, and DNA binding specificity is obtained by arranging one or more modules. Bibikova et al., 2001 Mol. Cell. Biol. 21:289-297; Boch et al., 2009 Science 326:1509-1512.

ZFNs can contain two or more (e.g., 2-8, 3-6, 6-8, or more) sequence-specific DNA binding domains (e.g., zinc finger domains) fused to an effector endonuclease domain (e.g., the FokI endonuclease). Porteus et al., 2005 Nat. Biotechnol. 23:967-973; Kim et al., 2007 Proceedings of the National Academy of Sciences of USA, 93:1156-1160; U.S. Pat. No. 6,824,978; PCT Publication Nos. WO1995/09233 and WO1994018313.

In one embodiment, the nuclease is a site-specific nuclease of the group or selected from the group consisting of omega, zinc finger, TALEN, and CRISPR/Cas.

The sequence-specific endonuclease of the methods and compositions described here can be engineered, chimeric, or isolated from an organism. Endonucleases can be engineered to recognize a specific DNA sequence, by, e.g., mutagenesis. Seligman et al. 2002 Nucleic Acids Research 30:3870-3879. Combinatorial assembly is a method where protein subunits form different enzymes can be associated or fused. Arnould et al. 2006 Journal of Molecular Biology 355:443-458. In certain embodiments, these two approaches, mutagenesis and combinatorial assembly, can be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the construct containing the one or more transgenes can be delivered by any method appropriate for introducing nucleic acids into a cell.

Guide RNA(s) used in the methods of the present disclosure can be designed so that they direct binding of the Cas-gRNA complexes to pre-determined cleavage sites in a genome. In one embodiment, the cleavage sites may be chosen so as to release a fragment or sequence that contains a region of a frame shift mutation. In further embodiment, the cleavage sites may be chosen so as to release a fragment or sequence that contains an extra chromosome.

For Cas family enzyme (such as Cas9) to successfully bind to DNA, the target sequence in the genomic DNA can be complementary to the gRNA sequence and may be immediately followed by the correct protospacer adjacent motif or “PAM” sequence. “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization and promote formation of a CRISPR complex. A target sequence may comprise any polynucleotide, such as DNA polynucleotides. The Cas9 protein can tolerate mismatches distal from the PAM. The PAM sequence varies by the species of the bacteria from which Cas9 was derived. The most widely used CRISPR system is derived from S. pyogenes and the PAM sequence is NGG located on the immediate 3′ end of the sgRNA recognition sequence. The PAM sequences of CRISPR systems from exemplary bacterial species include: Streptococcus pyogenes (NGG), Neisseria meningitidis (NNNNGATT), Streptococcus thermophilus (NNAGAA) and Treponema denticola (NAAAAC).

gRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81 , 82, 83, 84, 85, 86, 87, 88, 89, 90, 91 92, 93, 94, 95, 96, 97, 98, 99, or 100 nucleotides in length, or longer). In one embodiment, gRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate gRNA design, many computational tools have been developed (See Prykhozhij et al. 2015 PLoS ONE 10(3): Zhu et al. 2014 PLoS ONE 9(9); Xiao et al. 2014 Bioinformatics. Jan 21 (2014)); Heigwer et al. 2014 Nat Methods 11(2):122-123). Methods and tools for guide RNA design are discussed by Zhu 2015 Frontiers in Biology 10(4):289-296, which is incorporated by reference herein. Additionally, there is a publicly available software tool that can be used to facilitate the design of gRNA(s) (www.genscript.com/gRNA-design-tool) and https://www.idtdna.com/pages/products/crispr-genome-editing/alt-r-crispr-cas9-system).

Inhibitory Nucleic Acids that Hybridize to or Target HIF and KEAP1

In certain embodiments, the HIF inhibitor used in the present methods and compositions is a polynucleotide that reduces expression of HIF. Thus, the method involves administering an effective amount of a polynucleotide that specifically targets nucleotide sequence(s) encoding HIF. The polynucleotides reduce expression of HIF, to yield reduced levels of the gene product (the translated polypeptide).

The nucleic acid target of the polynucleotides (e.g., antisense oligonucleotides, and ribozymes) may be any location within the gene or transcript of HIF.

In further embodiments, the KEAP1 inhibitor used in the present methods and compositions is a polynucleotide that reduces expression of KEAP1. Thus, the method involves administering an effective amount of a polynucleotide that specifically targets nucleotide sequence(s) encoding KEAP1. The polynucleotides reduce expression of KEAP1, to yield reduced levels of the gene product (the translated polypeptide).

The nucleic acid target of the polynucleotides (e.g., antisense oligonucleotides, and ribozymes) may be any location within the gene or transcript of KEAP1.

The inhibitory nucleic acids may be RNA interference or RNAi, an antisense RNA, a ribozyme, or combinations thereof.

“RNA interference”, or “RNAi” is a form of post-transcriptional gene silencing (“PTGS”), and comprises the introduction of, e.g., double-stranded RNA into cells. The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. RNAi can work in human cells if the RNA strands are provided as pre-sized duplexes of about 19 nucleotide pairs, and RNAi worked particularly well with small unpaired 3′ extensions on the end of each strand (Elbashir et al. Nature 411:494-498 (2001)).

RNAi may be small interfering RNA or siRNAs, a small hairpin RNA or shRNAs, microRNA or miRNAs, a double-stranded RNA (dsRNA), etc.

The inhibitory nucleic acid may be a short RNA molecule, such as a short interfering RNA (siRNA), a small temporal RNA (stRNA), and a micro-RNA (miRNA). Short interfering RNAs silence genes through an mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

Software programs for predicting siRNA sequences to inhibit the expression of a target protein are commercially available and find use. One program, siDESIGN from Dharmacon, Inc. (Lafayette, Colo.), permits predicting siRNAs for any nucleic acid sequence, and is available on the internet at dharmacon.com. Programs for designing siRNAs are also available from others, including Genscript (available on the internet at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/. ”

RNA interference (RNAi) is a method of post transcriptional gene silencing (PTGS) induced by the direct introduction of double-stranded RNA (dsRNA) and has emerged as a useful tool to knock out expression of specific genes in a variety of organisms. RNAi is described by Fire et al., Nature 391:806-811 (1998). Other methods of PTGS are known and include, for example, introduction of a transgene or virus. Generally, in PTGS, the transcript of the silenced gene is synthesized but does not accumulate because it is rapidly degraded. Methods for PTGS, including RNAi are described, for example, in the Ambion.com world wide web site, in the directory “/hottopics/”, in the “rnai” file.

In some embodiments, the level of HIF is decreased in a desired target cell such as an RPE cell. Furthermore, in such embodiments, treatment may be targeted to, or specific to, desired target cell. The expression of HIF may be specifically decreased only in the desired target cell such as an RPE cell, and not substantially in cells. Thus, in such embodiments, the level of HIF, remains substantially the same or similar in non-target cells in the course of or following treatment.

In some embodiments, the level of KEAP1 is decreased in a desired target cell such as an RPE cell or RGC. Furthermore, in such embodiments, treatment may be targeted to, or specific to, desired target cell. The expression of KEAP1 may be specifically decreased only in the desired target cell such as an RPE cell or RGC, and not substantially in cells. Thus, in such embodiments, the level of KEAP1, remains substantially the same or similar in non-target cells in the course of or following treatment.

Alternately, one may administer the viral vectors, RNAi, shRNA or other inhibitor, or related compounds in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, specific neurons, or the vitreous, and more specifically hepatocytes. The liposomes will be targeted to and taken up selectively by the desired tissue. Also included in a targeted drug delivery system is nanoparticle specific delivery of the viral vectors, RNAi, shRNA or other HIF inhibitors, alone or in combination.

The inhibitory nucleic acids may be an antisense nucleic acid sequence that is complementary to a target region within the mRNA of HIF or KEAP1. The antisense polynucleotide may bind to the target region and inhibit translation. The antisense oligonucleotide may be DNA or RNA, or comprise synthetic analogs of ribo-deoxynucleotides. Thus, the antisense oligonucleotide inhibits expression of HIF or KEAP1.

An antisense oligonucleotide can be, for example, about 7, 10, 15, 20, 25, 30, 35, 40, 45, 50, 55, 60, 65, 70, 75, 80, or more nucleotides in length.

The antisense nucleic acid molecules of the invention may be administered to a subject or generated in situ such that they hybridize with or bind to the mRNA of HIF or KEAP1.

Ribozyme

The inhibitor may be a ribozyme that inhibits expression of the HIF or KEAP1 gene.

Ribozymes can be chemically synthesized and structurally modified to increase their stability and catalytic activity using methods known in the art. Ribozyme encoding nucleotide sequences can be introduced into host cells through gene-delivery mechanisms known in the art.

Antibodies

The present inhibitors can be an antibody or antigen-binding portion thereof that is specific to HIF or KEAP1.

The antibody or antigen-binding portion thereof may be the following: (a) a whole immunoglobulin molecule; (b) an scFv; (c) a Fab fragment; (d) an F(ab')2; and (e) a disulfide linked Fv. The antibody or antigen-binding portion thereof may be monoclonal, polyclonal, chimeric and humanized The antibodies may be murine, rabbit or human/humanized antibodies.

### Vectors

In some embodiments of the disclosure, the nucleic acids for use in the disclosed methods are contained in a vector, such as a viral vector. In some embodiments of the disclosure, the composition for use in the disclosed methods comprises a vector, such as a viral vector. The viral vector may be, for example, an AAV, adenovirus, lentivirus, retrovirus, poxvirus, baculovirus, herpes simplex virus, vaccinia virus, or a synthetic virus (e.g., a chimeric virus, mosaic virus, or pseudotyped virus, and/or a virus that contains a foreign protein, synthetic polymer, nanoparticle, or small molecule). The vector may also include conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide.

In some embodiments of the disclosure, the transgenes, e.g., PGC1α or NRF2, are operably linked to promoters that induce expression of the transgenes in the proper cells, e.g., RGC or RPE.

In some embodiments, nucleic acids inhibitors or encoding inhibitors, e.g., inhibitors of HIF, are linked to promoters to induce the expression of the nucleic acids in the proper cells, e.g., RPE.

In some embodiments, nucleic acids inhibitors or encoding inhibitors, e.g., inhibitors of KEAP1, are linked to promoters to induce the expression of the nucleic acids in the proper cells, e.g., RGC or RPE.

The promoter for targeting RGC may be, for example, hSNCG or Ple (NEFL).

The promoter for targeting RPE may be, for example, VMD2 or RPE65.

Alternatively, ubiquitous promoters may be used including without limitation, CMV, EF1, CAG, CB7, PGK and SFFV.

Other regulatory elements may also be used such as a polyadenylation sequence and post-transcriptional regulatory elements, for efficient pre-mRNA processing and increasing gene expression, respectively. For nucleic acids encoding proteins, a polyadenylation sequence generally is inserted following the transgene sequences. Examples of polyadenylation sequences include SV40, bGHpolyA and spA. Examples of post-transcriptional regulatory elements include WPRE, WPRE3 and HPRE.

In some embodiments, optimized combinations of polyadenylation sequences and post-transcriptional regulatory elements may be used in the vectors.

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

In some embodiments, the regulatory sequences impart tissue-specific gene expression capabilities. In some cases, the tissue-specific regulatory sequences bind tissue-specific transcription factors that induce transcription in a tissue specific manner

Recombinant AAV Vectors “Recombinant AAV (rAAV) vectors” described herein generally include a transgene (e.g., encoding PGC1α and NRF2). The transgene is flanked by 5′ and 3′ ITRs, and may be operably linked to one or more regulatory elements in a manner that permits transgene transcription, translation, and/or expression in a cell of a target tissue. Such regulatory elements may include a promoter or enhancer, such as the chicken beta actin promoter or cytomegalovirus enhancer, among others described herein. The recombinant AAV genome is generally encapsidated by capsid proteins (e.g., from the same AAV serotype as that from which the ITRs are derived or from a different AAV serotype from that which the ITRs are derived). The AAV vector may then be delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product of interest (e.g., PGC1α). Components of exemplary AAV vectors that may be used in conjunction with the compositions and methods of the disclosure are described below.

Any AAV serotype or combination of AAV serotype can be used in the methods and compositions of the present invention. Because the methods and compositions of the present invention are for the treatment and cure of mitochondrial disorders, AAV serotypes that target at least muscle, or at least muscle and the central nervous system can be used in some embodiments and include but are not limited to AAV1, AAV2, AAV4, AAV5, AAV6, AAV7, AAV8, and AAV9.

In some embodiments, AAV9 serotype, which has a wide tropism, is used.

In some embodiments, AAV2 serotype is used.

In some embodiments, AAV8 serotype is used.

Components of AAV Vectors

The AAV vectors described herein may contain cis-acting 5′ and 3′ ITRs (See, e.g., Carter, in “Handbook of Parvoviruses”, ed., P. Tijsser, CRC Press, pp. 155 168 (1990)). The ITR sequences are typically about 145 bp in length. Preferably, substantially the entire sequences encoding the ITRs are used in the molecule, although some degree of minor modification of these sequences is permissible. (See, e.g., texts such as Sambrook et al, (1989) and Fisher et al., (1996)). An example of such a molecule is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In addition to the elements identified above for recombinant AAV vectors, the vector may also include conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA).

For nucleic acids encoding proteins, a polyadenylation sequence generally is inserted following the transgene sequences and before the 3′ AAV ITR sequence. An rAAV construct useful in the present invention may also contain an intron, desirably located between the promoter/enhancer sequence and the transgene. One possible intron sequence is derived from SV-40, and is referred to as the SV-40 T intron sequence.

Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available (see, e.g., Sambrook et al, and references cited). Such a motif may be useful, for example, for instances in which multiple genes or portions thereof are expressed from the same AAV vector.

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

Examples of constitutive promoters include, without limitation, a chicken beta actin promoter, a retroviral Rous sarcoma virus (RSV) LTR promoter (optionally with a RSV enhancer), a cytomegalovirus (CMV) promoter (optionally with a CMV enhancer), a SV40 promoter, a dihydrofolate reductase promoter, a 13-actin promoter, a phosphoglycerol kinase (PGK) promoter, and an EFla promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system (WO 98/10088); a ecdysone insect promoter (No et al., Proc. Natl. Acad. Sci. USA 93:3346-3351 (1996)), a tetracycline-repressible system (Gossen et al., Proc. Natl. Acad. Sci. USA 89:5547-5551 (1992)), a tetracycline-inducible system (Gossen et al., Science 268:1766-1769 (1995), a RU486-inducible system (Wang et al., Nat. Biotech. 15:239-243 (1997) and Wang et al., Gene Ther. 4:432-441 (1997)) and a rapamycin-inducible system (Magari et al., J. Clin. Invest. 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, a native promoter, or fragment thereof, for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner

In some embodiments, one or more bindings sites for one or more of miRNAs are incorporated in a transgene of a rAAV vector, to inhibit the expression of the transgene in one or more tissues of a subject harboring the transgenes. The miRNA target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

For example, a 3′UTR site which would inhibit the expression of the transgene in the liver can be incorporated into a transgene. This would be beneficial for transgenes which encode therapeutic proteins which are toxic to the liver as most of the virus administered (approximately 60 to 90%) is eventually found in the liver. Thus suppressing the therapeutic gene expression in liver relieves the burden from liver cells.

In some embodiments, the AAV vector will be modified to be a self-complementing AAV. A self-complementing AAV carries complementary sequence of the transgene (i.e., a double copy of the transgene). Self complementation makes the gene more stable after it enters the cell.

Methods of Obtaining of AAV Vectors

Methods for obtaining recombinant AAVs having a desired capsid protein have been described (See, for example, U.S. Pat. No. 7,906,111). A number of different AAV capsid proteins have been described, for example, those disclosed in Gao, et al., J. Virology 78(12):6381-6388 (June 2004); Gao, et al., Proc Natl Acad Sci USA 100(10):6081-6086 (May 13, 2003); and U.S. Pat. No. 7,906,111; U.S. Pat. No. 8,999,678. For the desired packaging of the presently described constructs and methods, the AAV9 vector and capsid, or the AAV2 vector and capsid, is preferred. However, it is noted that other suitable AAVs such as rAAVrh.8 and rAAVrh.10, or other similar vectors may be adapted for use in the present compositions. Typically the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters.

The recombinant AAV vector, rep sequences, cap sequences, and helper functions for producing the rAAV may be delivered to the packaging host cell using any appropriate genetic element (vector). The selected genetic element may be delivered by any suitable method, including those described herein. See, e.g., Fisher et al, J. Virology 70:520-532 (1993) and U.S. Pat. No. 5,478,745.

In some embodiments, recombinant AAVs may be produced using the triple transfection method (e.g., as described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present invention include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Exemplary Recombinant AAV Compositions

The current disclosure provides for compositions containing a recombinant AAV containing a nucleic acid sequence that encodes a functional protein. Such proteins include, without limitation, PGC1α or NRF2.

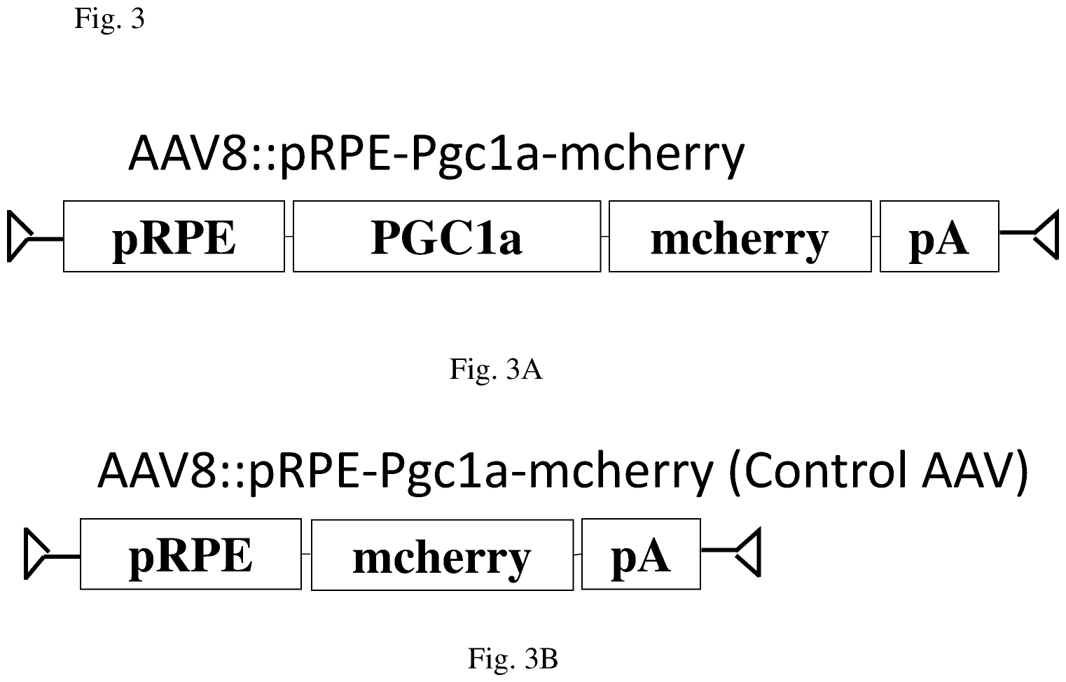

An exemplary recombinant AAV to deliver PGC1α to RPE is shown in FIG. 3.

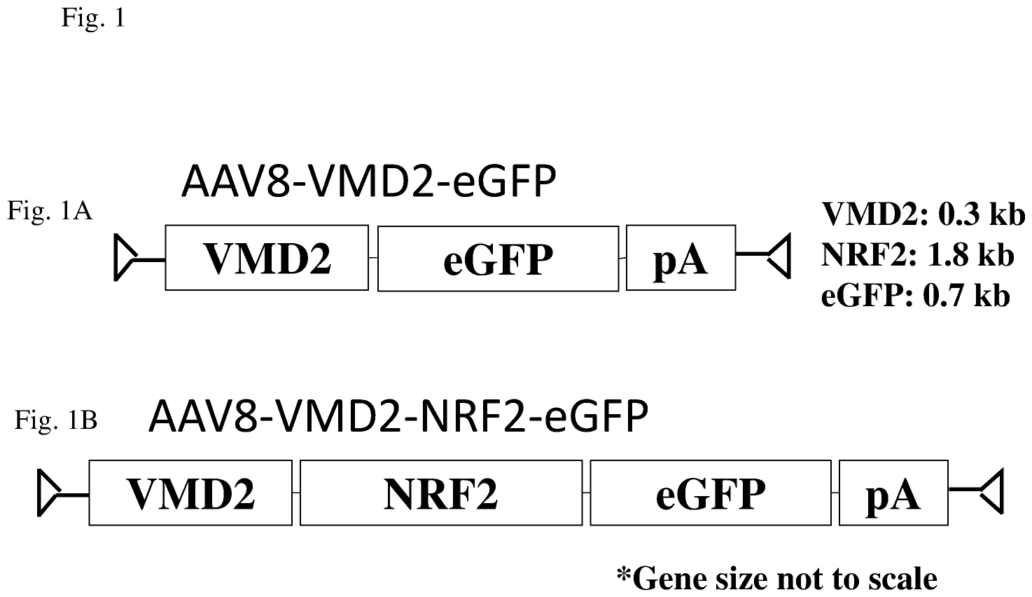

An exemplary recombinant AAV to deliver NRF2 to RPE is shown in FIG. 1.

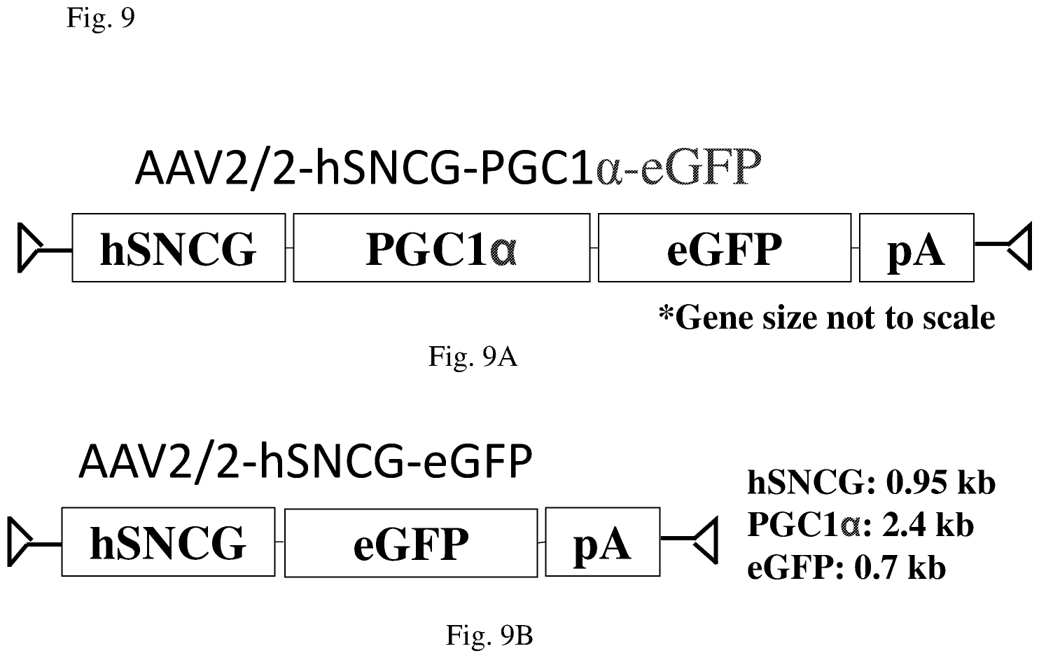

An exemplary recombinant AAV to deliver PGC1α to RGC is shown in FIG. 9.

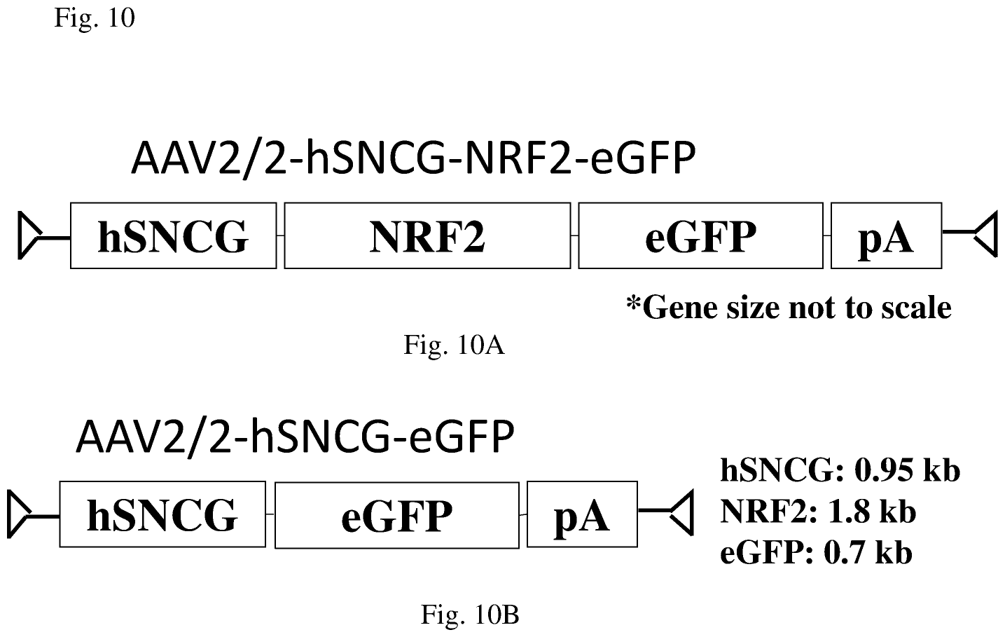

An exemplary recombinant AAV to deliver NRF2 to RGC is shown in FIG. 10.

### Pharmaceutical Compositions, Routes of Administration and Dosing

The current disclosure provides vectors, e.g., AAV and lentiviral, for use in methods of delaying the onset of, treating, preventing, and/or curing a neurodegenerative disease or disorder including but not limited to glaucoma, retinitis pigmentosa (RP), age-related macular degeneration (AMD), autosomal dominant optic atrophy (ADOA), Alzheimer's disease, Parkinson's disease, Huntington's disease, Amyotrophic lateral sclerosis (ALS), and Lewy body dementia, and/or alleviating in a subject at least one of the symptoms associated with these diseases.

In some embodiments, methods involve administration of a rAAV vector that encodes one or more peptides, polypeptides, endonucleases, gRNAs, shRNAs, microRNAs, or antisense nucleotides, in a pharmaceutically-acceptable carrier to the subject in an amount and for a period of time sufficient to delay the onset of, treat, prevent and/or cure the neurodegenerative disease or disorder including but not limited to glaucoma, retinitis pigmentosa (RP), age-related macular degeneration (AMD), autosomal dominant optic atrophy (ADOA), Alzheimer's disease, Parkinson's disease, Huntington's disease, Amyotrophic lateral sclerosis (ALS), and Lewy body dementia in the subject having or suspected of having such a disorder.

The rAAVs may be delivered to a subject in compositions according to any appropriate methods known in the art. The rAAV, preferably suspended in a physiologically compatible carrier (e.g., in a composition), may be administered to a subject. In certain embodiments, compositions may comprise a rAAV alone, or in combination with one or more other viruses (e.g., a second rAAV encoding having one or more different transgenes or an endonuclease).

In one embodiment, a composition can comprise an rAAV2/2 vector comprising a nucleic acid sequence comprising a transgene encoding a functional protein including but not limited to PGC1α and a hSNCG promoter.

In one embodiment, a composition can comprise an rAAV2/2 vector comprising a nucleic acid sequence comprising a transgene encoding a functional protein including but not limited to PGC1α and a Ple (NEFL) promoter.

In one embodiment, a composition can comprise an rAAV2/2 vector comprising a nucleic acid sequence comprising a transgene encoding a functional protein including but not limited to NRF2 and a hSCNG promoter.

In one embodiment, a composition can comprise an rAAV2/2 vector comprising a nucleic acid sequence comprising a transgene encoding a functional protein including but not limited to NRF2 and a Ple (NEFL) promoter.

In a further embodiment, a composition can comprise an rAAV8 vector comprising a nucleic acid sequence comprising a transgene encoding a functional protein including but not limited to PGC1α and a VMD promoter.

In a further embodiment, a composition can comprise an rAAV8 vector comprising a nucleic acid sequence comprising a transgene encoding a functional protein including but not limited to NRF2 and a VMD promoter.

In a further embodiment, a composition can comprise an rAAV8 vector comprising a nucleic acid sequence comprising a transgene encoding a functional protein including but not limited to PGC1α and an RPE65 promoter.

In a further embodiment, a composition can comprise an rAAV8 vector comprising a nucleic acid sequence comprising a transgene encoding a functional protein including but not limited to NRF2 and an RPE65 promoter.

In one embodiment, a composition can comprise an rAAV8 vector comprising an inhibitor or a nucleic acid sequence encoding an inhibitor of HIF, and a VMD2 promoter.

In one embodiment, a composition can comprise an rAAV8 vector comprising an inhibitor or a nucleic acid sequence encoding an inhibitor of KEAP1, and a VMD2 promoter.

In one embodiment, a composition can comprise an rAAV8 vector comprising an inhibitor or a nucleic acid sequence encoding an inhibitor of HIF, and an RPE65 promoter.

In one embodiment, a composition can comprise an rAAV8 vector comprising an inhibitor or a nucleic acid sequence encoding an inhibitor of KEAP1, and an RPE65 promoter.

In one embodiment, a composition can comprise an rAAV2/2 vector comprising an inhibitor or a nucleic acid sequence encoding an inhibitor of KEAP1, and a hSCNG promoter.

In some embodiments, methods involve administration of a lentiviral vector that encodes one or more peptides, polypeptides, endonucleases, gRNAs, shRNAs, microRNAs, or antisense nucleotides, in a pharmaceutically-acceptable carrier to the subject in an amount and for a period of time sufficient to delay the onset of, treat, prevent and/or cure the neurodegenerative disease or disorder including but not limited to glaucoma, retinitis pigmentosa (RP), age-related macular degeneration (AMD), autosomal dominant optic atrophy (ADOA), Alzheimer's disease, Parkinson's disease, Huntington's disease, Amyotrophic lateral sclerosis (ALS), and Lewy body dementia in the subject having or suspected of having such a disorder.

The lentiviral vectors may be delivered to a subject in compositions according to any appropriate methods known in the art. The lentiviral vector, preferably suspended in a physiologically compatible carrier (e.g., in a composition), may be administered to a subject. In certain embodiments, compositions may comprise a lentiviral vector alone, or in combination with one or more other viruses (e.g., a viral vector encoding having one or more different transgenes or an endonuclease).

In one embodiment, a composition can comprise a lentiviral vector comprising an inhibitor or a nucleic acid sequence encoding an inhibitor of HIF, and a VMD2 promoter.

In one embodiment, a composition can comprise a lentiviral vector comprising an inhibitor or a nucleic acid sequence encoding an inhibitor of HIF, and an RPE65 promoter.

In one embodiment, a composition can comprise a lentiviral vector comprising an inhibitor or a nucleic acid sequence encoding an inhibitor of KEAP1, and a VMD2 promoter.

In one embodiment, a composition can comprise a lentiviral vector comprising an inhibitor or a nucleic acid sequence encoding an inhibitor of KEAP1, and an RPE65 promoter.

In one embodiment, a composition can comprise a lentiviral vector comprising an inhibitor or a nucleic acid sequence encoding an inhibitor of KEAP1, and a hSCNG promoter.

Suitable carriers may be readily selected by one of skill in the art in view of the indication for which the rAAV or lentiviral vector is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV or lentiviral vector and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In some embodiments, rAAV compositions are formulated to reduce aggregation of AAV particles in the composition, particularly where high rAAV concentrations are present (e.g., about 10¹³ GC/ml or more). Methods for reducing aggregation of rAAVs are well known in the art and, include, for example, addition of surfactants, pH adjustment, and salt concentration adjustment (see, e.g., Wright, et al., Molecular Therapy 12:171-178 (2005).

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1 or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active rAAV or lentiviral vector in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In addition to the methods of delivery described above, the following techniques are also contemplated as alternative methods of delivering the compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations, transdermal matrices (U.S. Pat. Nos. 5,770,219 and 5,783,208) and feedback-controlled delivery (U.S. Pat. No. 5,697,899).

rAAVS and lentiviral vectors are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected tissue (e.g., intracerebral administration, intrathecal administration), intravenous, oral, inhalation (including intranasal and intratracheal delivery), intraocular, intravenous, intramuscular, subcutaneous, intradermal, and other parental routes of administration. Routes of administration may be combined, if desired. The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

The present invention provides stable pharmaceutical compositions comprising rAAV virions. The compositions remain stable and active even when subjected to freeze/thaw cycling and when stored in containers made of various materials, including glass.

Appropriate doses will depend on the subject being treated (e.g., human or nonhuman primate or other mammal), age and general condition of the subject to be treated, the severity of the condition being treated, the mode of administration of the rAAV virions, among other factors. An appropriate effective amount can be readily determined by one of skill in the art.

The dose of rAAV virions required to achieve a desired effect or “therapeutic effect,” e.g., the units of dose in vector genomes/per kilogram of body weight (vg/kg), will vary based on several factors including, but not limited to: the route of rAAV administration; the level of gene or RNA expression required to achieve a therapeutic effect; the specific disease or disorder being treated; and the stability of the gene or RNA product. One of skill in the art can readily determine a rAAV virion dose range to treat a subject having a particular disease or disorder based on the aforementioned factors, as well as other factors that are well known in the art. An effective amount of the rAAV is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the rAAV, and the route of administration. In some cases, a dosage between about 10¹⁰ to 10¹² rAAV genome copies per subject is appropriate. In certain embodiments, 10¹² rAAV genome copies per subject is effective to target desired tissues. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg.

Thus, a “therapeutically effective amount” will fall in a relatively broad range that can be determined through clinical trials. For example, for in vivo injection, i.e., injection directly to the subject, a therapeutically effective dose will be on the order of from about 10⁵ to 10¹⁶ of the rAAV virions, more preferably 10⁸ to 10¹⁴ rAAV virions. For in vitro transduction, an effective amount of rAAV virions to be delivered to cells will be on the order of 10⁵ to 10¹³, preferably 10⁸ to 10¹³ of the rAAV virions. If the composition comprises transduced cells to be delivered back to the subject, the amount of transduced cells in the pharmaceutical compositions will be from about 10⁴ to 10¹⁰ cells, more preferably 10⁵ to 10⁸ cells. The dose, of course, depends on the efficiency of transduction, promoter strength, the stability of the message and the protein encoded thereby, etc. Effective dosages can be readily established by one of ordinary skill in the art through routine trials establishing dose response curves.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ rAAV virions in a single dose, or two, four, five, six or more doses that collectively result in delivery of, e.g., 10⁵ to 10¹⁶ rAAV virions. One of skill in the art can readily determine an appropriate number of doses to administer.

Pharmaceutical compositions will thus comprise sufficient genetic material to produce a therapeutically effective amount of the protein of interest, i.e., an amount sufficient to reduce or ameliorate symptoms of the disease state in question or an amount sufficient to confer the desired benefit. Thus, rAAV virions will be present in the subject compositions in an amount sufficient to provide a therapeutic effect when given in one or more doses. The rAAV virions can be provided as lyophilized preparations and diluted in the virion-stabilizing compositions for immediate or future use. Alternatively, the rAAV virions may be provided immediately after production and stored for future use.

The pharmaceutical compositions will also contain a pharmaceutically acceptable excipient or carriers. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences and U.S. Pharmacopeia: National Formulary, Mack Publishing Company, Easton, Pa. (1984).

Formulations of therapeutic and diagnostic agents may be prepared by mixing with acceptable carriers, excipients, or stabilizers in the form of, e.g., lyophilized powders, slurries, aqueous solutions or suspensions.

Toxicity and therapeutic efficacy of the therapeutic compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

In certain embodiments, route of administration is subretinal injection, intravitreal injection or suprachoroidal injection.

Doses can be adjusted to optimize the effects in the subject. Additionally, a subject can be monitored for improvement of their condition prior to increasing the dosage.

### Kits

The present disclosure also provides kits comprising the components of the combinations disclosed herein in kit form. A kit of the present disclosure includes one or more components including, but not limited to, viral vectors (e.g., AAV vectors or lentiviral vectors) described herein. Kits may further include a pharmaceutically acceptable carrier, as discussed herein. The viral vector can be formulated as a pure composition or in combination with a pharmaceutically acceptable carrier, in a pharmaceutical composition.

In some embodiments, a kit includes an AAV vector containing a transgene described herein in one container (e.g., in a sterile glass or plastic vial). In some embodiments, a kit includes an AAV or lentiviral vector containing an inhibitor described herein in one container (e.g., in a sterile glass or plastic vial).

If the kit includes one or more pharmaceutical compositions for subretinal injection or intravitreal injection to a subject, the kit can include a device for performing such administration.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Targeted AAV Vector Delivers NRF2 and Enhances Antioxidant Response in RPE which Promotes Cone and Rod Survival

### Materials and Methods

### RP Mouse Model

Pde6b^{H620Q} mice (Pde6b^{H620Q} homozygotes), a well established preclinical RP mouse model, harbor a missense mutation in the phosphodiesterase 6 catalytic domain, undergo normal photoreceptor differentiation, and retain retinal function before degeneration onset. ERG has been used to show that Pde6b^{H620Q} mice lost rod responses at postnatal day (P)30 and cone responses after P60. Histology of retinal sections showed 4 rows of nuclei at P28 and 2 rows at P49. Compared with Pde6b^{rd1} mice, Pde6b^{H620Q} mice exhibit relatively delayed photoreceptor degeneration, beginning as early as 2-3 weeks after birth and with near-complete loss of photoreceptors by 3 months (Davis, et al. 2008).

### AAV Vector Construction

To enhance antioxidant responses in RPE, a VMD2 upstream region from −253 to +38 bp (approximately 300 bp) is used as an RPE-specific promoter (Esumi, et al. 2004) to deliver mouse Nrf2 (1.8 kb) (FIG. 1). Murine Nrf2 was reported to contain 2 nuclear localization signal (NLS) motifs that can shuttle Nrf2 into the nucleus (Theodore, et al. 2008). Two vectors are generated: AAV8-VMD2-eGFP as a control for validation and AAV8-VMD2-NRF2-eGFP to test our hypothesis (FIG. 1). As a negative control, RPE is transduced with AAV8-VMD2-eGFP.

### Subretinal Injection

The procedure is performed as previously described (Davis, et al. 2008; Wang, et al. 2013). In these procedures, the eyelids of 1 eye of a newborn mouse is opened artificially using microsurgery scissors. Approximately 1 μl of virus solution (10¹² GC/ml) is injected subretinally into the right eye on postnatal day 5 (P5). The left eye serves as a negative control. These procedures have previously resulted in 30%-50% retinal cell transduction.

The control vector is injected first and the expression of GFP limited to the RPE is seen. Then the AAV8-VMD2-NRF2-eGFP is injected into the mice.

The mice are evaluated as follows:
Immunoblot. Protein is extracted from RPE and probed with monoclonal antibodies against NQO1 (SC-16464; Santa Cruz), HO-1 (SC-10789; Santa Cruz) and mouse beta actin antibody (ab8224; Abcam) as a loading control.
RNA extraction and real-time qPCR. RNA is extracted from RPE and reverse-transcribed. Real-time qPCR is performed using primers for Nrf2 target genes NQO1, HO-1, GCLC, GCLM, and glutathione synthetase.
ERGs. ERGs is performed as described (Davis, et al. 2008; Wang, et al. 2010). Briefly, recordings are made using Espion ERG Diagnosys equipment (Diagnosys LLL, Lowell, Mass.). Responses are averaged for each trial. The dim light scotopic b-wave is measured to assess rod-specific function, the scotopic maximal b-wave to assess inner retina function, the scotopic maximal a-wave to assess photoreceptor-specific function, and the photopic b-wave to assess cone-specific function. ERGs is performed at P30 and P60 for Pde6b^{H620Q} mice.
Spectral domain optical coherence tomography (SD-OCT) live-imaging quantification of photoreceptors. Photoreceptor survival is quantified via live imaging, and measuring of rod and cone outer segment lengths. Briefly, ONL thickness is determined using readings from SD-OCT imaging with Spectralis equipment (Heidelberg, Germany) located in the Columbia core mouse facility.
Immunohistochemistry and fluorescent staining. Tissues are collected at P60 for Pde6b^{H620Q} mice Immunolabeling and fluorescent staining on retinal cryosections and neuroretina flat mounts are performed as described previously (Wang, et al. 2013; Tosi, et al. 2010) with the following modifications. One eye per animal is processed for cryosectioning; the other is dissected into neuroretina for flat mounts. The eyecups for cryosectioning are fixed in a cold 4% paraformaldehyde PBS solution overnight, whereas neuroretina is flat-mounted and fixed for 1 h. The following primary and secondary antibodies and dilutions are used: rabbit anti-arr3 (cone arrestin 1:1,000; ab15282; Millipore); mouse anti-acrolein (1:250; ab48501; Abcam) for lipid peroxidation; rabbit α-VDAC (1:1000; D73D12; Cell Signaling) for mitochondrial marker; goat anti-rabbit (1:1,000; A-11034; Invitrogen). Nuclei are counterstained with DAPI.
Quantification of rod survival. Rhodopsin mRNA in the retina is measured as another measure of the numbers of rods. The expression of Cyclophilin is stable under many different conditions, and will be used as a standard for normalization of rhodopsin by real time RT-PCR.
Quantification of cone survival. Quantification of the number of cones is performed using cryosections and neuroretina flat-mounts from both eyecups, as described. Ten serial sections are incubated with anti-Arr3 antibodies. Nuclei are counterstained with DAPI. All images are acquired with the Nikon Ti Eclipse inverted confocal microscope in our Confocal and Specialized Microscopy Core Facility. Arrestin⁺ cells are counted using semiautomatic quantitative ImageJ software.
Experimental Comparisons. Scotopic maximal and photopic b-wave amplitudes (2 time points), and ONL thickness (1 time point) between the right eye (injected with AAV8-VMD2-NRF2-eGFP) and the left (injected with control AAV) in Pde6b^{H620Q} mice are compared. This design allows the use paired t tests. Left-right eye differences between different virus groups will be assessed.

### Results

AAV8-VMD-eGFP transduction allows the specific expression of GFP in RPE. Thus, the injections of the AAV8-VMD2-NRF2-eGFP vector in the mouse models of RP is performed.

The overexpression of NRF2 in RPE slows rod and cone degeneration, as is evident from ERG analysis and histology. Additionally, the upregulation of ARE-related genes in RPE collected from AAV8-VMD2-NRF2-eGFP injected eyes relative to AAV8-VMD2-eGFP injected eyes or non-injected eyes in the RP mouse model is also seen.

### Example 2—Targeted AAV Vector Delivered PGC1α and Enhanced Antioxidant Response in RPE which Promoted Cone and Rod Survival

### Materials and Methods

The same materials and methods were used as in Example 1 for the RP mouse model, and subretinal injection and assessments including oxidative phosphorylation markers, ERG, and immunohistochemistry and fluorescent staining with anti-an antibody, and the experimental comparison.

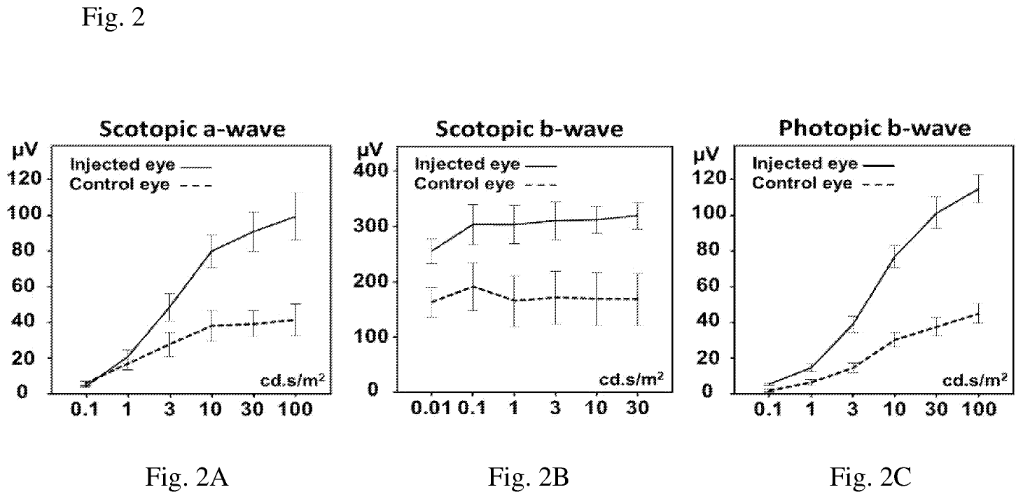

A recent study in a mouse model of retinitis pigmentosa reported a detrimental effect of retinal PGC la overexpression. A potential weakness with this in vivo mouse study is that it used the non-specific CMV promoter, which led to nonspecific overexpression in RPE and photoreceptors. However, in our in vivo mouse study, PGC1α overexpression driven by RPE promoter showed significantly protecting photoreceptors in our RP mouse model (FIG. 2). This indicates that driving PGC1α overexpression with the CMV promoter may reprogram metabolism differently in RPE and photoreceptors, which may cause unanticipated cellular damage to the photoreceptors. Therefore, there is a need for a cell-specific targeted therapy.

As discussed, to enhance antioxidant responses in RPE, a VMD2 upstream region from −253 to +38 bp (approximately 300 bp) as an RPE-specific promoter (Esumi, et al. 2004) was used to deliver mouse PGC1α (FIG. 3). Two vectors were generated: AAV8::RPE65-mcherry as a control for validation (FIG. 3B) and AAV8::RPE65-PGC1α-mcherry to test our hypothesis (FIG. 3A).

For controls, the left eye of the mice were not injected.

### Results

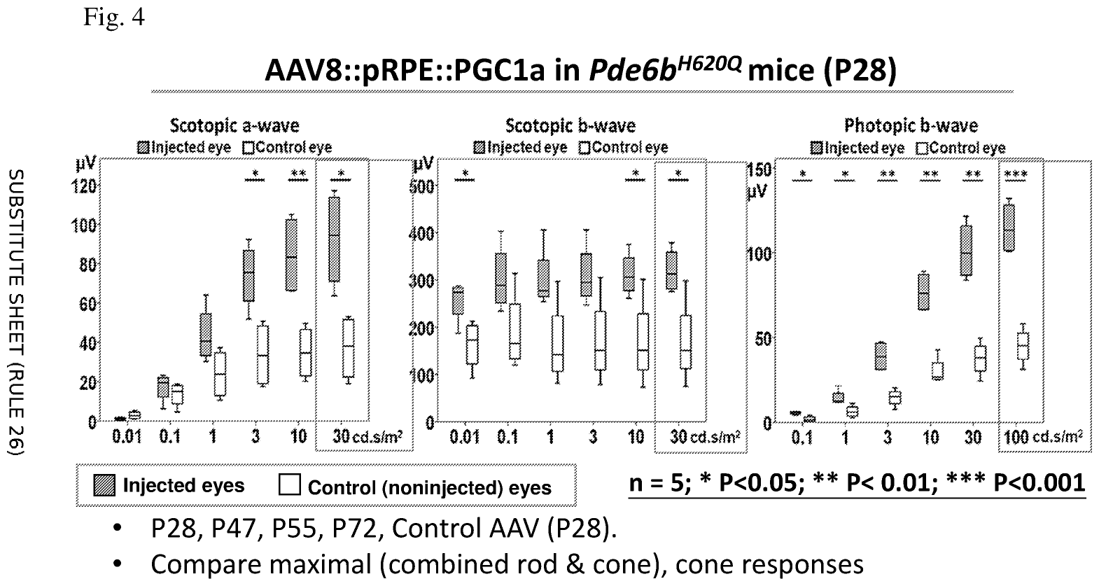

The results of ERG showed rescue at one month old (FIG. 4).

The serial intensities in scotopic a wave and b waves, and photopic b waves were compared. The white boxes represent the injected right eye, and empty boxes represent the noninjected control left eye. There were statistically significant difference between injected and noninjected eyes at some intensities in scotopic responses and all intensities in photopic responses.

Similar results were seen at P28, P47, P55, P72 as well as control AAV at P28.

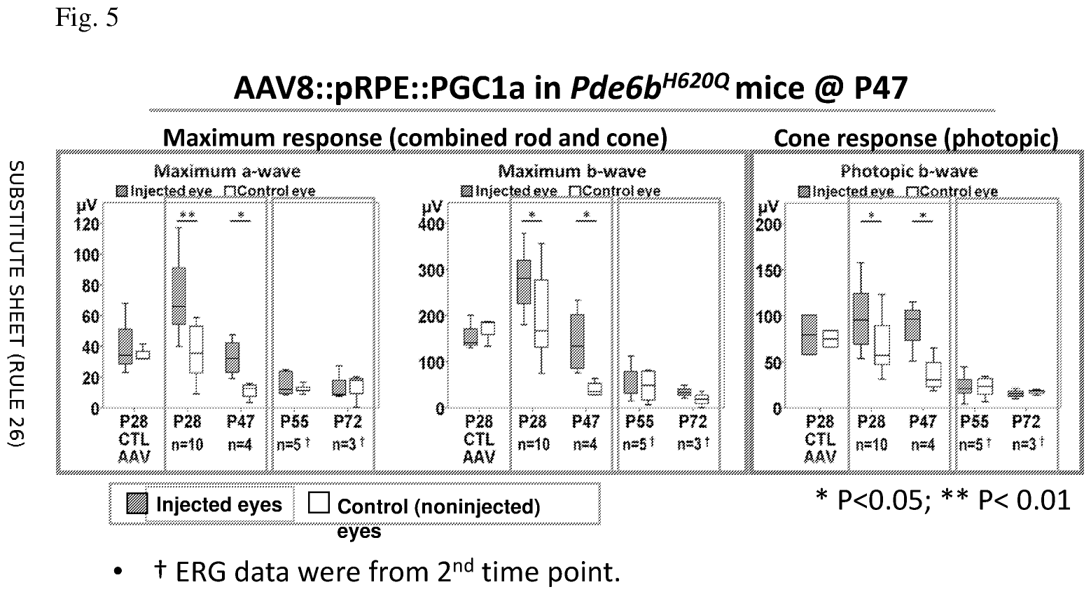

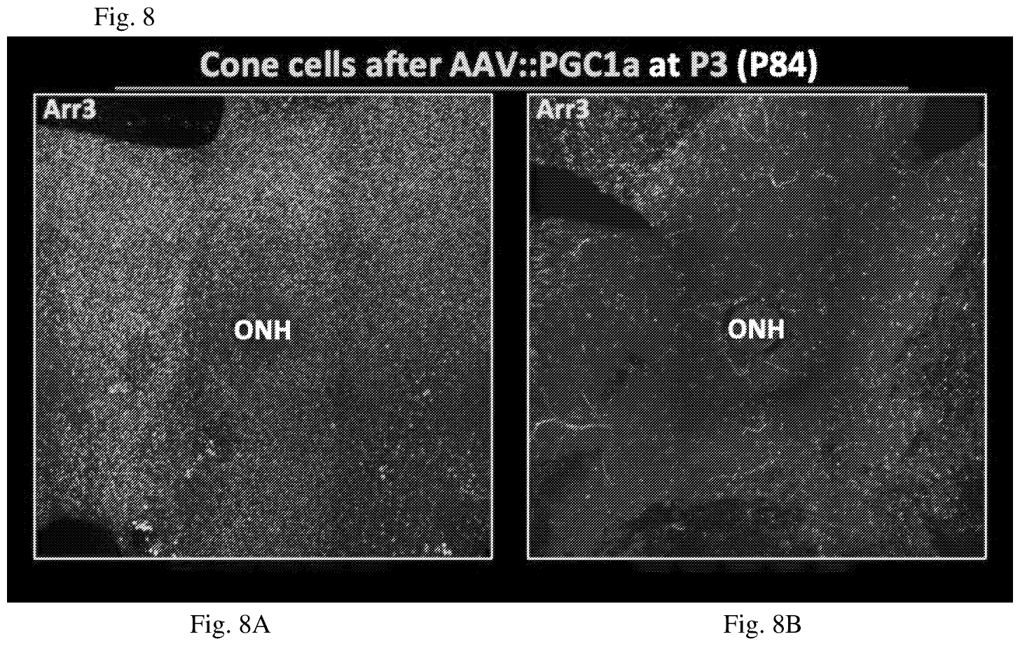

For comparison, maximal responses (which is combined rod and cone) and cone responses were compared (FIG. 5). The a- and b-waves of maximum response and b-waves of cone response at 4 different time points and control AAV virus at P28 were compared in the first lane of FIG. 8. There was statistically significant difference between the injected and noninjected eyes at P28 and P47, but not at P55 and P72. The ERG results at the P55 and P72 were from mice that had been tested by ERG at either earlier time points. Some of these mice developed corneal opacity after the first ERG testing, which affected the ERG amplitude.

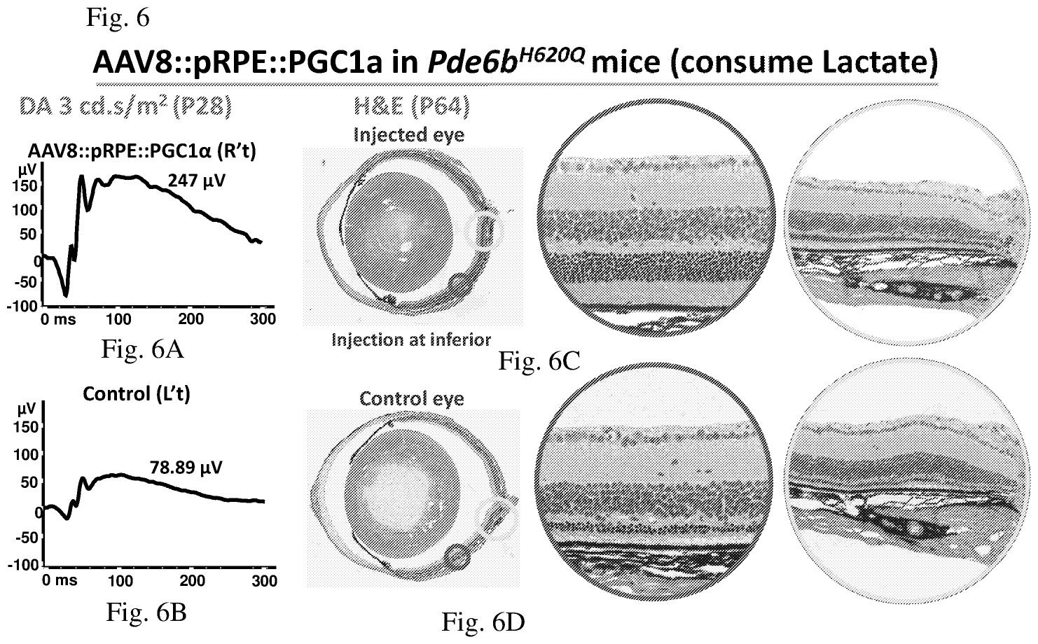

FIG. 6 shows the results of one mouse with functional and anatomical rescue. The scotopic ERG at one month old showed larger amplitudes in the injected right eye (FIG. 6A) as compared to the amplitudes in the control non-injected left eye (FIG. 6B), indicating preserved photoreceptor function B.

FIGS. 6C and 6D show H&E staining of these two eyes at two months old. There are more outer nuclear layers in the injected eye compared to fellow eye. This difference was not caused by the retinal folds, detachment or eyeball deformity and could be observed around the optic nerve head. Thus, the histology at P64 showed a deceleration in photoreceptor degeneration in the injected eye as compared to the control eye, revealing higher levels of photoreceptor survival in the AAV8-PGC1α injected eye.

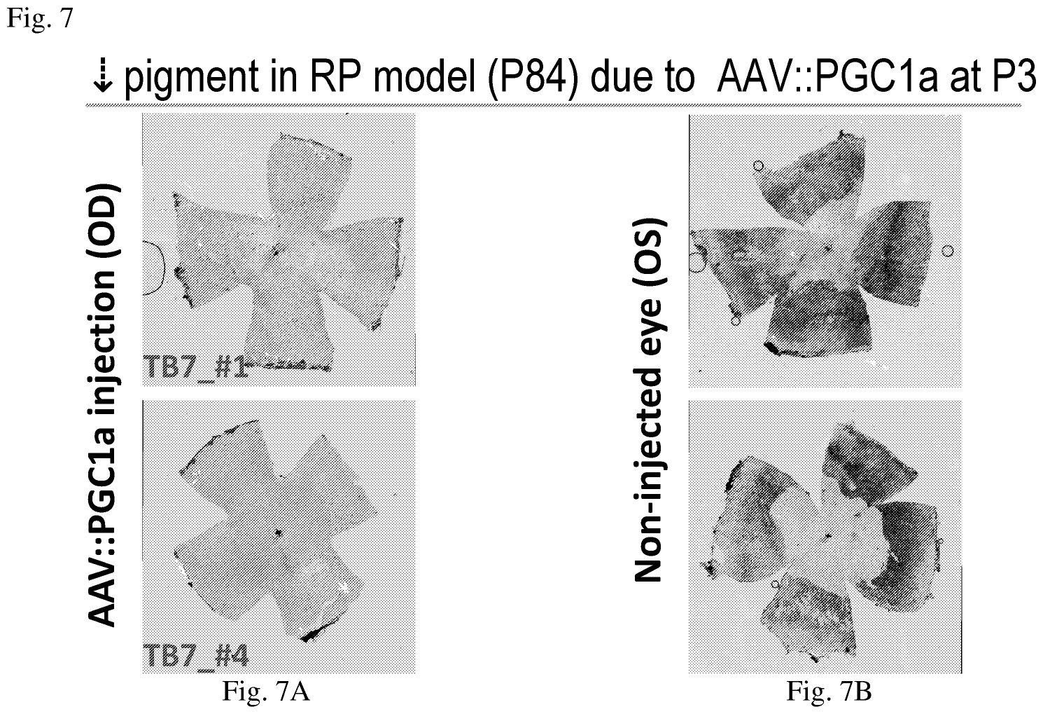

FIG. 7 shows whole mount retina in the injected eye (FIG. 7A) and the noninjected eye (FIG. 7B) in a mouse at 3 months old. It is very clear that the whole mount retina in the injected eye has less pigment migration compared to the noninjected eye. The pigment migration is a sign of severe photoreceptor degeneration as is seen in the RP patients.

As shown in FIG. 8, there are also more cone specific labeled Arr3 cells in the injected eye as compared to the noninjected eye.

These results show that the targeted administration of PGC1α to RPE decreased photoreceptor degeneration in a mouse model of RP.

### Example 3—Enhancing an Antioxidant Response in RPE by Conditional Overexpression of NRF2 and Conditional Knockout of KEAP Promotes Cone and Rod Survival

### Materials and Methods

### RPE-Specific Inducible Cre Recombinase Line (Rpe65^{CreERT2} Mice)

An inducible RPE-specific Cre recombinase line (Rpe65^{CreERT2} mice) in which Cre is controlled by the endogenous naïve Rpe65 locus and stably expressed over the mouse's lifetime was generated. Unlike other RPE-Cre drivers, Rpe65^{CreERT2} mice harbor a Cre-estrogen receptor (CreER-T2) fusion construct. Specifically, Cre recombinase (Cre) is fused to a mutant estrogen ligand-binding domain (ERT2) that requires tamoxifen for activity; the T2 variant binds tamoxifen with higher affinity than estrogen, and Cre activity is not detectable in control females. Upon tamoxifen induction, Cre^{ERT2} translocates to the nucleus, where it interacts with loxP sites, and then returns to the cytoplasm, minimizing the chance of unintended mutagenesis. Crossing these mice with Ai75D reporter mice (JAX #25106) results in tdTomato expression in RPE cell nuclei after tamoxifen induction. Inducible Rpe65^{CreERT2} mice can be used to determine whether enhancing antioxidant response by either overexpressing NRF2 or knocking out KEAP1, respectively, in RPE promotes photoreceptor survival in RP.

### Enhancing Antioxidant Response in RPE by Conditional Overexpressing NRF2

To enhance antioxidant response in RPE, R26^{LSL-nrf2/+} mice were generated that conditionally overexpress mouse NRF2 in RPE when crossed with Rpe65^{CreERT2} mice. These crosses generate control R26^{+/+}; Rpe65^{CreERT2/+} and experimental R26^{LSL-Nrf2/+}; Rpe65^{CreERT2/+} genotypes. The R26^{LSL-Nrf2/+}; Rpe65^{CreERT2/+} line is also crossed with Pde6b^{H620Q} mice.

### Enhancing Antioxidant Response in RPE by Conditional Knockout of KEAP1

The main control of NRF2 stability is exerted by KEAP1. Therefore, to enhance antioxidant response in RPE, we will take advantage of C57BL/6-Keap1^{tm2.1Brsp} mice (Taconic Biosciences, Model 8799; hereafter Keap1^{f/f}) that conditionally knockout KEAP1 and cause subsequently NRF2 activation in RPE when crossed with Rpe65^{CreERT2} mice. These crosses generate control Keap1^{+/+}; Rpe65^{CreERT2/+} and experimental Keap1^{f/f}; Rpe65^{CreERT2/+} genotypes. The Keap1^{f/f}; Rpe65^{CreERT2/+} line is also crossed with Pde6b^{H620Q} mice.

RPE from eye cups of tamoxifen-inducted R26^{LSL-Nrf2/+}; Rpe65^{CreERT2/+}, Keap1^{f/f}; Rpe65^{CreERT2/+} and Rpe65^{CreERT2/+} mice are isolated after incubation in trypsin at 37° C., 5% CO₂ for 45 min (Fernandez, et al. 2016), and immunoblots and qPCR as described in Example 1 are performed.

Additionally, the following assays are performed to evaluated photoreceptor function and survival as described in Example 1: ERGs; spectral domain optical coherence tomography (SD-OCT) live-imaging quantification of photoreceptors; immunohistochemistry and fluorescent staining; quantification of rod survival; and quantification of cone survival.

Amplitudes of scotopic maximal b-waves, photopic b-waves (2 timepoints, P30 and P60), and ONL thickness (1 timepoint, P60) are compared between experimental R26^{LSL-nrf2/+}; Rpe65^{CreERT2/+} and control R26^{+/+}; Rpe65^{CreERT2/+} genotypes; experimental Keap1^{f/f}; Rpe65^{CreERT2/+} and control Keap1^{+/+}; Rpe65^{CreERT2/+} genotypes in Pde6b^{H620Q} mouse RP model.

Glutamate is important as a metabolite because it is required for synthesis of glutathione. Isolation of glutamate from the oxidative pathway relies on cytosolic NADH/NAD⁺. Additional experiments, such as dihydroethidium staining for detection of superoxide (Zhang, et al. 2013), GSH/GSSG (#26406, Cayman Chemical) (Williams, et al. 2017) and NAD+/NADH quantitation Colorimetric kit (K337, Biovision) (Williams, et al. 2017) are performed to define Redox state.

### Results

ARE-related genes (NQO1, HO-1, GCLC, GCLM, and glutathione synthetase) in the isolated RPE from tamoxifen-inducted R26^{LSL-Nrf2/+}; Rpe65^{CreERT2/+}, Keap1^{f/f}; Rpe65^{CreERT2/+} mice are upregulated. Enhancing antioxidant response by overexpressing NRF2 in RPE promotes photoreceptor survival and function in the mouse model of RP. Enhancing antioxidant response by conditional knockout of KEAP1 in RPE promotes photoreceptor survival and function in the mouse model of RP as well. The neuroprotective effect by conditional overexpressing NRF2 are similar to or slightly less than conditional knockout KEAP1 because NRF2 turns over rapidly and is found at low levels due to constant degradation by the ubiquitin proteasome system (McMahon, et al. 2004).

### Example 4—Determination if OXPHOS/Catabolism in RPE Promotes Cone and Rod Survival by Conditional Overexpression and Conditional Knockout of Pgc1α

### Materials and Methods

### RPE-Specific Inducible Cre Recombinase Line (Rpe65^{CreERT2} Mice)

The same mice used in Example 3 are used to determine whether enhancing or suppressing OXPHOS by either overexpressing or knocking out Pgc1α, respectively, in RPE promotes photoreceptor survival in RP.

### Enhancing OXPHOS in RPE

To enhance OXPHOS in RPE, Rosa26^{LSL-Pgc1α/+} mice are generated that conditionally overexpress mouse Pgc1α in RPE when crossed with Rpe65^{CreERT2} mice. These crosses generate control Rosa26^{+/+}; Rpe65^{CreERT2/+} and experimental Rosa26^{LSL-Pgc1α/+}; Rpe65^{CreERI2/+} genotypes. The Rosa26^{LSL-Pgc1α/+}; Rpe65^{CreERT2/+} line is also crossed with the Pde6b^{H620Q} and Rho^{D190N/+} RP models. Although Rho^{D190N/+} mice have a knock-in mutation in the Rho gene, which, like the Rosa26 gene, is located on Chromosome 6, this strategy is still feasible

### Suppressing OXPHOS in RPE

OXPHOS is suppressed by PGC1α knockout and use of a conditional Pgc1α knockout mice (Ppargc1α^{tm2.1Brsp}/J, fAX #9666; or Pgc1α^{f/f}) for this experiment. Pgc1α^{f/f} mice are be crossed through 2 generations with Rpe65^{CreERT2} mice to generate control Pgc1+^{+/+}; Rpe65^{CreERT2/+} and experimental Pgc1α^{f/f}; Rpe65^{CreERT2/+} genotypes. To investigate the effect of suppressing OXPHOS in RPE during retinal degeneration, Pgc1α^{f/f}; Rpe65^{CreERT2/+} line is crossed with the Rho^{D190N/+} RP model and all mice are genotyped to confirm the absence of the rd8 and rd1 mutations.

RPE from eye cups of tamoxifen-inducted R26^{LSL-Nrf2/+}; Rpe65^{CreERT2/+} Keap1^{f/f}; Rpe65^{CreERT2/+} and Rpe65^{CreERT2/+} mice are isolated after incubation in trypsin at 37° C., 5% CO₂ for 45 min (Fernandez, et al. 2016), and immunoblots, qPCR, glucose uptake assay and U-13C glucose Flux are performed as follows:
Immunoblot. Protein is extracted from RPE and probed with 5 monoclonal antibodies against subunits of OXPHOS complexes (Complex I NDUFB8, Complex II SDHB, Complex III UQCRC2, Complex IV MTCO1, and Complex V ATP5A) using the Total OXPHOS Rodent WB Antibody Cocktail Kit (ab110413; Abcam) and rabbit anti-alpha tubulin antibody (ab4074; Abcam) as a loading control.
RNA extraction and real-time qPCR. RNA is extracted from RPE and reverse-transcribed. Real-time qPCR is performed using primers for PPARA (which regulates genes required for fatty acid utilization); ESRRA, NRF1, and GABPA (all of which regulate OXPHOS genes); and NRE2L2 and FOXO3 (which regulate antioxidant genes). For OXPHOS subunits, primers for ATPSO, a component of the F1-FO ATPase; COX4I1 and COXSB, components of complex IV; and NDUFBS, a component of complex I are used.
Glucose uptake assay. Retinas are dissected in cold DMEM and cultured in DMEM with or without D-glucose in the presence of the fluorescent glucose analog 2-deoxy-D-glucose (2-NBDG, 1 mM), washed 4 times with ice-cold PBS and DAPI, flat-mounted between 2 pre-chilled cover slides, and imaged immediately.
U-¹³C glucose flux. Cultured RPE from experimental Rosa26^{LSL-Pgc1α/+}; Rpe65^{CreERT2/+} are incubated with U-¹³C glucose to monitor flux through glycolysis and the citric acid cycle, or incubated with U-¹³C glutamine to specifically monitor oxidative flux. The RPE are harvested and homogenized and extracted in chloroform/methanol and analyzed by GC/MS as previously described (Zhang, et al. 2016).

Additionally, the following assays are performed to evaluated photoreceptor function and survival as described in Example 1: ERGs; and spectral domain optical coherence tomography (SD-OCT) live-imaging quantification of photoreceptors.

For the enhancement of OXPHOS, experimental comparisons of the amplitudes of scotopic maximal b-waves, photopic b-waves (2 timepoints), and ONL thickness (1 timepoint) between experimental Rosa26^{LSL-Pgc1α/+}; Rpe65^{CreERT2/+} and control Rosa26^{+/+}; Rpe65^{CreERT2/+} genotypes in Pde6b^{H620Q} and Rho^{D190N/+} mouse RP models are performed.

For the suppression of the OXPHOS, experimental comparisons of the amplitudes of scotopic maximal b-waves (2 timepoints), photopic b-wave (2 timepoints), and ONL thickness (1 timepoint) between the experimental Pgc1α^{f/f}; Rpe65^{CreERT2/+} and control Pgc1α^{+/+}; Rpe65^{CreERT2/+} genotypes using the Rho^{D190N/+} mouse RP model are performed.

### Results

Enhancing OXPHOS after PGC1α overexpression in RPE promotes photoreceptor survival and function in a mouse model of RP. Inhibiting PGC1α in RPE accelerates photoreceptor degeneration further supporting the hypothesis.

### Example 5—Targeted AAV Vector Delivers PGC1α or NRF2 and Restores Mitochondrial Function in RGCs

### Materials and Methods

### Glaucoma Mouse Model

DBA/2J mice (pigmentary glaucoma mouse model, hereafter referred to as D2 mice) are an inbred strain D2 is homozygous for the glaucoma-related Gpnmb^{R150X} and Tyrp1^{isa} mutations. The D2 mouse is widely used in glaucoma research, as it phenocopies human pigmentary glaucoma and develops iris pigment dispersion at 5˜6 months, increased intraocular pressure at 6˜8 months, and RGC death at 10˜12 months (John, et al. 1998). The PERG in DBA/2J mice is impaired early in the disease, preceding loss of retinal nerve fiber layer thickness (Saleh, et al. 2007; Howell, et al. 2007).

Viral Vectors to Overexpress PGC1α and NRF2 in RGCs using an RGC-Specific Promoter

Although AAV2/2 has been shown to transduce RGCs more efficiently than any other cell type in the retina, promoter choice is important for cell-specific and transgenic expression within the targeted therapy. Recent studies demonstrated efficient gene therapy in a model of ADOA and pigmentary glaucoma by delivering Opa1 and Nmnat1, a gene encoding a key NAD(+)-producing enzyme, under the control of the cytomegalovirus (CMV) promoter. Although the CMV promoter and a hybrid CMV early enhancer/chicken β-actin promoter (CAG) have been shown to facilitate transduction of about 85% of RGCs in the adult rat eye, with similar expression seen in mice (Nickells, et al. 2017; Martin, et al. 2003; Harvey, et al. 2002) the preliminary data with AAV intravitreal injection driven by the CMV promoter showed that other types of retinal cells were also transduced (results not shown). Therefore, careful consideration needs to be taken to ensure that off-target transgene expression does not have adverse effects on other retinal cells.

PGC1α function in the retina is controversial and has been little studied in vivo (Xiong, et al. 2015). A recent study in a mouse model of retinitis pigmentosa reported a detrimental effect of retinal PGC1α overexpression (Xiong, et al. 2015). A potential weakness with this in vivo mouse study is that it used the CMV promoter, which led to nonspecific overexpression.

In addition, using the CMV promoter would likely force constitutive NRF2 activation in other cells in a nonphysiological manner that could lead to unanticipated complications because nuclear accumulation of NRF2 might reduce apoptosis and promote oncogenesis and drug resistance.

The hypothesis shown herein is that enhancing mitochondrial biogenesis only in RGCs could be a potential therapy for diseases with RGC death, such as glaucoma and ADOA. The approach is to test preclinical gene therapy with an RGC-specific promoter to target mitochondrial biogenesis in the two mouse models described above-models of RGC death. An RGC-specific promoter to drive PGC1α and NRF2 expression in order to determine its role in mitochondrial biology in RGCs.

To enhance mitochondrial biogenesis in RGCs, the hSncg (human gamma-synuclein) upstream region from −785 to +163 bp (approximately 948 bp) as an RGC-specific promoter (Chaffiol, et al. 2017) to deliver mouse Ppargc1α (Pgc1α) (2.4 kb) is used (FIG. 9A). The hSNCG promoter (948 bp) is a smaller, stronger, and more specific RGC promoter compared with the Thy1 promoter (6,500 bp), allowing PGC1α to be packaged together in this AAV2/2-hSNCG-PGC1α-eGFP vector. Two vectors are generated: AAV2/2-hSNCG-eGFP, as a control for validation, and AAV2/2-hSNCG-PGC1α-eGFP, to test the hypothesis (FIG. 9B). As a negative control, RGCs will be transduced with AAV2/2-hSNCG-eGFP.

To enhance antioxidant responses in RGCs, a hSncg promoter to deliver mouse Nrf2 (1.8 kb) is used (FIG. 14). Murine Nrf2 was reported to contain 2 nuclear localization signal motifs that can shuttle Nrf2 into the nucleus (Theodore, et al. 2008). Two vectors are generated: AAV2/2-hSNCG-eGFP as a control for validation and AAV2/2-hSNCG-NRF2-eGFP to test the hypothesis (FIG. 10). As a negative control, RGCs are transduced with AAV2/2-hSNCG-eGFP.

Intravitreal injection of the vectors into the mice is performed using a procedure modified from subretinal injections, as previously described (Wang, et al. 2010; Davis, et al.

2008). Four-week-old mice were anesthetized by intraperitoneal injection of ketamine/xylazine when needed, as described previously. Approximately 1.5 μl of virus solution (2×10⁷ transducing units [TU]/m1) were injected intravitreally into the right eye at the limbus by inserting a blunt-ended glass pipette through the puncture hole at a 45° angle, through the sclera into the vitreous body. The left eye served as a negative control. Intravitreal injections using these protocols have previously resulted in diffuse retinal transduction using the CMV promoter.

The control vectors AAV2/2-hSNCG-eGFP and AAV2/2-hSNCG-eGFP are injected first and the expression of GFP limited to the RGCs is seen. The AAV2/2-hSNCG-PGC1α-eGFP and AAV2/2-hSNCG-NRF2-eGFP are injected into the mice.

The mice are evaluated as follows:
Pattern electroretinograms (PERGs). PERGs is performed as described (Chou, et al. 2018; Porciatti 2013). Briefly, recordings are made using Celeris Diagnosys equipment (Diagnosys LLC, Lowell, Mass.). At the beginning of each session, negative control mice (adult C57BL/6J) are tested before testing treated mice. Responses are averaged for each trial. The P1N2 amplitude from P1 to N2 is measured to assess RGC-specific function. PERGs is performed at P180 to P360 for D2 mice.
Photopic negative responses (PhNR). PERGs is assessed as described (Chrysostomou and Crowston 2013). Briefly, photopic responses to 6 different stimulus strengths between 0.34 and 2.22 log cd·s/m² are presented on a 40-cd·s/m² rod-saturating green background. At each intensity, 25 flashes with an interstimulus interval of 3,000 ms are averaged. To assess RGC-specific function, PhNR amplitudes from the baseline to the PhNR trough (BT) are measured. PERGs are performed at P180 to P360 for D2 mice.
Immunohistochemistry and fluorescent staining. Tissues are collected at P180 and P360 for D2 mice Immunolabeling and fluorescent staining on retinal cryosections and neuroretinal flat mounts will be performed as described previously (Wang, et al. 2010; Tosi, et al. 2010) with the following modifications: One eye per animal is processed for cryosectioning; the other is dissected into neuroretina for flat mounts. Eyecups for cryosectioning are fixed in a cold 4% paraformaldehyde PBS solution overnight, whereas neuroretinas to be flat-mounted are fixed for 1 h. The following primary and secondary antibodies and dilutions are used: goat anti-Brn3 (1:200; SC-6026; Santa Cruz), rabbit anti-RBPMS (1:500; NBP2-20112; Novus Biologicals); donkey anti-goat (1:1,000; A-21432; Invitrogen), donkey anti-rabbit (1:1,000; A-31572; Invitrogen). Nuclei are counterstained with DAPI.
Quantification of RGC survival. RGC numbers are quantified using cryosections and neuroretina flat-mounts from both eyecups, as described (Wang, et al. 2010; Tosi, et al. 2010). Ten serial sections are incubated with anti-Brn3 antibodies. Nuclei are counterstained with DAPI. All images are acquired with the Nikon Ti Eclipse inverted confocal microscope located in our Confocal and Specialized Microscopy Core Facility. Brn3⁺ cells are counted using semiautomatic quantitative ImageJ software.
Noninvasive spectral domain optical coherence tomography (SD-OCT) live-imaging quantification of photoreceptors. RGC survival is quantified via live imaging and measure the thickness of the ganglion cell complex using readings from SD-OCT imaging with Spectralis equipment (Heidelberg, Germany) located in the Columbia core mouse facility.
Mitochondrial DNA. Relative copy numbers of mtDNA and nuclear DNA in RGCs are determined by quantitative PCR, as described (Malik, et al. 2016). Specific primers were designed for the mitochondrial 16S rRNA gene and the nuclear β2 microglobulin gene, as described. A standard dilution series was used to confirm the efficiency of exponential amplification for each primer pair.
Electron microscopy to evaluate mitochondrial morphology and axonal myelination. Optic nerves from mice are processed as described (Wang, et al. 2013). Ultrathin sections are taken transversely through the optic nerve, stained with uranyl acetate and lead citrate, and images are photographed using a Hitachi 7100 TEM (Hitachi, Tokyo, Japan) equipped with an AMT digital camera.
Experimental comparisons. The amplitudes of PhNR (BT) and PERG (P1N2) at 2 timepoints (P180 and P360), and RGC numbers at 1 timepoint (P360) between the right eye (injected with AAV2/2-hSNCG-PGC1α-eGFP or AAV2/2-hSNCG-NRF2-eGFP) and the left (no injection) in D2 mice are compared. To improve scientific rigor, AAV2/2-hSNCG-eGFP is injected in the right eye in an additional group as a control. This design will allow us to use paired t tests.

### Results

AAV2/2-hSNCG-eGFP transduction allows the specific expression of GFP in RGCs. Thus, the injections of the AAV2/2-hSNCG-PGC1α-eGFP or AAV2/2-hSNCG-NRF2-eGFP vectors in the mouse models of RGC death is performed.

As hSNCG or Ple (NEFL) are RGC-specific promoters, PGC1α or NRF2 expression is limited to RGCs and that RGC-specific expression of the Pgc1α or Nrf2 gene delays RGC death.

RNA and protein are extracted from RGCs and immunoblotting using a MitoBiogenesis WB Antibody Cocktail Kit and real-time qPCR is performed to calculate the relative expression of mitochondrial biogenesis—related genes and mitophagy-related genes. An upregulation of mitochondrial biogenesis genes and proteins in RGCs collected from AAV vector—injected eyes (AAV2/2-hSNCG-PGC1α-eGFP) relative to controls is seen.

With regard to the AAV2/2-hSNCG-NRF2-eGFP vector injected eyes, RGC-specific expression of the Nrf2 gene delays RGC death. FACS is used to sort eGFP+RGCs and then analyze ARE-related genes and define the redox status of the RGCs. Up-regulation of ARE-related genes in RGCs collected from AAV2/2-hSNCG-NRF2-eGFP injected eyes relative to AAV2/2-hSNCG-eGFP injected eyes in 2 different RGC death mouse models is seen.

### Example 6 —Determination if Mitochondrial Biogenesis in RGC Promotes RGC Survival by Conditional Overexpression of and Conditional Knockout of Pgc1α

### Materials and Methods

### Conditional Overexpression Rosa26^{LSL-PGC1α/+} Mouse Line

PGC1α is a master regulator of mitochondrial biogenesis and has been shown to induce mitochondrial biogenesis in hippocampal neurons. Thus, to enhance mitochondrial biogenesis in vivo a knock-in mouse line was generated that conditionally overexpresses mouse Pgc1α. To create this line, a CAG-loxP-STOP-loxP-Pgc1α-IRES-GFP-polyA cassette was inserted into a Rosa26 gene-targeting plasmid (CTV plasmid, a gift of Dr. Klaus Rajewsky). Expression of PGC1α protein is suppressed until a lox-transcriptional stop-lox cassette (LSL) is excised by CRE. Mouse lines were generated by homologous recombination in KV1 (129S6/SvEvTac×C57BL/6J) embryonic stem (ES) cells, followed by injection of targeted ES cells into C57BL/6J blastocysts. Chimeras were bred for germline transmission. Mice were backcrossed to a C57BL/6J background for at least 6 generations. Fibroblasts from a Rosa26^{LSL-Pgc1α/+} mouse transfected with AAV8-Cre were GFP-positive and overexpressed PGC1α, indicating that the Rosa26^{LSL-Pgc1α/+} mouse overexpresses PGC1α when the STOP cassette is removed after crossing with a Cre recombinase line. To target PGC1α overexpression in the vast majority of RGCs, Tg(Thy1-cre/ER^{T2},-EYFP)HGfng/PyngJ mice (JAX #12708; hereafter Thy1^{CreERT2} mice) was used to target Cre recombinase to RGCs.

### Suppress Mitochondrial Biogenesis in RGCs by Conditional Knockout of PGC1α

We will take advantage of conditional Pgc1α knockout mice (Ppargc1α^{tm2.1Brsp}/J, JAX #9666; or Pgc1α^{f/f}) for this experiment. Pgc1α^{f/f} mice are be crossed through 2 generations with Thy1^{CreERT2/+} mice to generate control Pgc1α^{+/+}; Thy1^{CreERT2/+} and experimental Pgc1α^{f/f}; Thy1^{CreERT2/+} genotypes. To investigate the effect of suppressing mitochondrial biogenesis in RGCs during degeneration, the Pgc1α^{f/f}; Thy1^{CreERT2/+} line is crossed with D2 mice. All mice will be genotyped to confirm the absence of the rd8 and rd1 mutations, which may affect photoreceptor function (Mattapallil, et al. 2012; Errjgers, et al. 2007).

### Enhance Mitochondrial Biogenesis in RGCs by Conditional Overexpression of PGC1α

Rosa26^{LSL-Pgc1α/+} mice have been generated that conditionally overexpress mouse Pgc1α in RGCs when crossed with Thy1^{CreERT2} mice. These crosses generate control Rosa26^{+/+}; Thy1^{CreERT2/+} and experimental Rosa26^{LSL-Pgc1α/+}; Thy1^{CreERT2/+} genotypes. Because D2 mice carry homozygous Gpnmb^{R150X} on Chromosome 6 (where the knock-in PGC1α in the Rosa26 gene is also located), crossing D2 and Rosa26^{LSL-Pgc1α/+}; mice is not feasible.

Mouse retinas are dissociated by enzymatic digestion followed by filtration using Falcon 70 μm nylon strainers, as described (Williams, et al. 2017; Chitalapud, et al. 2017; Chintalapudi, et al. 2016), and then the following assays are performed:
Fluorescence-activated cell sorting (FACS) of RGCs. Anti-mouse CD16/32 antibody (clone 93; BioLegend) is used to minimize nonspecific binding of antibodies to cells expressing Fcγ receptors II and III. To detect surface antigens, cells are incubated on ice for 30 min with the following cocktail of primary antibodies: anti-CD90.2 Alexa Fluor-700 (Thy1.2, clone 30-H12, BioLegend); anti-CD48 PE-Cy7 (clone HM48-1, BioLegend, labels monocytes and microglia); anti-CD15 PE (clone MC-480, BioLegend, labels amacrine cells); and anti-CD57 (clone VC1.1, Sigma Aldrich, St. Louis, Mo.; also labels amacrine cells). Because the anti-CD57 antibody is unconjugated, a Brilliant Violet 421-tagged secondary antibody (clone Poly4053, BioLegend) is used to allow for sorting. This cocktail has allowed accurate removal of other retinal cell types during FACS. Thy1.2⁺CD48^{neg}CD15^{neg}CD57^{neg} cells will be sorted and frozen at −80° C. until further processing.
Immunoblots. Protein is extracted from live sorted Thy1.2⁺CD48^{negCD}15^{neg}CD57^{neg} cells and probed with 2 monoclonal antibodies against subunits of OXPHOS enzyme complexes, Complex II SDH-A (nuclear DNA-encoded) and Complex IV (COX-1, mtDNA-encoded) using the MitoBiogenesis WB Antibody Cocktail Kit (ab123545; Abcam).The mouse anti-beta-actin antibody (ab8224; Abcam) is used as a loading control.
RNA extraction and real-time qPCR. RNA is extracted from Thy1.2⁺CD48^{neg}CD15^{neg}CD57^{neg} cells and reverse transcribed. Real-time qPCR is performed using primers for the mitochondrial biogenesis-related genes PolgA and Tfam and for the mitophagy-related genes Pink1, Park2, Bnip3l, and Lc3b.

The same tests done as described in Example 5 including PERGs, PhNR, immunohistochemistry and fluorescent staining, quantification of RGC survival, SD-OCT, testing of mitochondrial DNA, and electron microscopy are done to evaluate RGC function and survival.

PhNR (BT) and PERG (P1N2) (2 timepoints, P180 and P360) amplitudes and RGC numbers (1 timepoint, P360) between experimental and control genotypes in D2 mice using 2 different strategies (suppression and enhancement of mitochondrial biogenesis) are compared.

### Results

Enhancing mitochondrial biogenesis by PGC1α overexpression in RGCs enhances RGC survival and function in a mouse model of RGC death. Inhibiting PGC1α in RGCs accelerates RGC degeneration. PGC1α overexpression delays RGC death and increases the number of mitochondria in RGCs and in the optic nerve.

### Example 7—Determination if Mitochondrial Biogenesis in RGC Promotes RGC Survival by Conditional Overexpression and Conditional Knockout of NRF2 and Conditional Knockout of KEAP1

### Materials and Methods

### Conditional Overexpression Rosa26^{LSL-Nrf2/+} Line

NRF2 is a key nuclear transcription factor for the systemic antioxidant defense system. Thus, to enhance the antioxidant response in vivo, a knock-in mouse line was generated that conditionally overexpresses mouse Nrf2 using the same strategy as with the Rosa26^{LSL-Nrf2/+} mice. Chimeras were bred for germline transmission. Mice were backcrossed to a C57BL/6J background for at least 6 generations. To validate the conditional overexpression of NRF2 in Rosa26^{LSL-Nrf2/+} mice, Rosa26^{LSL-Nrf2/+} was crossed to Pde6g^{CreERT2/+} to generate Rosa26^{LSL-Nrf2/+}; Pde6g^{CreERT2/+} mice. After tamoxifen induction, protein was extracted from retinas and probed with anti-NRF2 antibody (1:1,000; HPA003097; MilliporeSigma). Immunoblotting revealed increased NRF2 expression (results not shown), indicating that the Rosa26^{LSL-Nrf2/+} mouse overexpressed NRF2 when the STOP cassette was removed after crossing with a Cre recombinase line. To target NRF2 overexpression in the vast majority of RGCs, Thy1^{CreERT2} mice was used to target Cre recombinase in RGCs.

### Suppress the Antioxidant Response in RGCs by Conditional Knockout of NRF2

Conditional Nrf2 knockout mice (Nfe2l2^{tm1.1Sred}/SbisJ, JAX #25433; or Nrf2^{f/f}) are used for this experiment. Nrf2^{f/f} mice are crossed through 2 generations with Thy1^{CreERT2/+} mice to generate control Nrf2^{+/+}; Thy1^{CreERT2/+} and experimental Nrf2^{f/f};Thy1^{CreERT2/+} genotypes. To investigate the effect of suppressing the antioxidant response in degenerating RGCs, the Nrf2^{f/f}; Thy1^{CreERT2/+} line is crossed with the Opa1^{V346D/+} and D2 mice. All mice are genotyped to confirm the absence of the rd8 and rd1 mutations (Mattapallil, et al. 2012; Errjgers, et al. 2007).

### Enhance the Antioxidant Response in RGCs by Conditional Overexpression of NRF2

Rosa26^{LSL-Nrf2/+} mice were generated that conditionally overexpress mouse NRF2 in RGCs when crossed with Thy1^{CreERT2} mice. These crosses generate control Rosa26^{+/+}; Thy1^{CreERT2/+} and experimental Rosa26^{LSL-Nrf2/+}; Thy1^{CreERT2/+} genotypes. Because D2 mice carry homozygous Gpnmb^{R150X}, located on Chromosome 6, this strategy is not feasible in the D2 strain.

### Enhance Antioxidant Response in RGCs by Conditional Knockout of Keap1

NRF2 stability is largely controlled by the E3 ligase adapter KEAP1. Therefore, to enhance the antioxidant response in RGCs, we will take advantage of C57BL/6-Keap1^{tm1.1Mri} mice (Taconic Biosciences, Model 8799; hereafter Keap1^{f/f}) in which the Keap1 gene is conditionally knocked out, causing subsequent Nrf2 activation in RGCs when crossed with Thy1^{CreERT2} mice. These crosses generate mice with the control Keap1^{+/+};Thy1^{CreERT2/+} and experimental Keap1^{f/f}; Thy1^{CreERT2/+} genotypes. The Keap1^{f/f}; Thy1^{CreERT2/+} line are also crossed with D2 mice and genotype all mice to confirm the absence of the rd8 and rd1 mutations (Mattapallil, et al. 2012; Errjgers, et al. 2007).

Mouse retinas are dissociated by enzymatic digestion as described in Example 6 and the evaluations as described in Example 6 are performed.

### Results

Inhibiting NRF2 in RGCs accelerates RGC death in the model of glaucoma. Enhancing the antioxidant response by overexpression of NRF2 or knockout of KEAP1 in RGCs promoted RGC survival and function in a mouse model of ADOA. These results are comparable in both mouse models of RGC death, even though the NRF2 overexpression strategy (knock-in Rosa26 gene) is not feasible in D2 mice because they carry homozygous GpnmbR150X on chromosome 6, where the Rosa26 gene is also located. ARE-related genes (NQO1, HO-1, GCLC, GCLM, and glutathione synthetase) are down-regulated in the FACS-sorted RGCs following Nrf2 knockout in 2 different RGC death mouse models and that ARE-related genes are up-regulated in the NRF2 overexpression mouse model of ADOA.

### Example 8—Ablation of HIF in RPE Promoted Cone and Rod Survival

### Materials and Methods

A Pde6b^{H620Q/H620Q}; Hif-2α^{tm1Mcs}/Hif-2α^{tm1Mcs} mouse was generated as follows.

Three lines of mice were crossed to develop the breeding strains. Hif-2α^{tm1Mcs}/Hif-2α^{tm1Mcs} Jax Stock #008407 mice were purchased from the Jackson Laboratory. Pde6b^{H620Q}/Pde6b^{H620Q} mice were rederived via oviduct transfer using European Mouse Mutant Archive (EMMA) morulae (Davis, et al. 2008; Hart, et al. 2005); and Rpe65^{CreERT2} mice described in Example 3. All mice were housed in the Columbia University Pathogen-free Eye Institute Annex Animal Care Services Facility and maintained with a 12-h light/12-h dark cycle.

Pde6b^{H620Q/H620Q} mice were crossed with Rpe65^{CreERT2} mice, and their offspring were bred with Hif-2α^{tm1Mcs}/Hif-2α^{tm1Mcs} Jax mice. Six generations of backcrosses were required to generate breeding mice. The resulting progeny were homozygous for all alleles of interest (Pde6b, Hif2, and Rpe65), but some were wild type at Rpe65, whereas others possessed the Rpe65^{CreERT2} mutation. We isolated these two lines for use as breeding strains. Crossing the breeding strains produced the experimental mice, which are homozygous at the Pde6b and Hif2 loci, and heterozygous at the Rpe65 locus. At P7, half of the experimental mice were given a 100 μg/g body weight (BW) injection of tamoxifen (100 mg/ml in ethanol; catalog T5648; Sigma-Aldrich), which was diluted with corn oil to a concentration of 10 mg/ml and thoroughly mixed at 42° C. One injection was administered on P7, P8, and P9. The other half of the experimental mice were injected with ethanol (10% in corn oil) following the same dosage as tamoxifen and served as the control group. There was no discrimination based on the sex of the mice.

The Columbia University Institutional Animal Care and Use Committee (IACUC) approved all experiments prior to initiation. Mice were used in accordance with the Statement for the Use of Animals in Ophthalmic and Vision Research of the Association for Research in Vision and Ophthalmology and the Policy for the Use of Animals in Neuroscience Research of the Society for Neuroscience.

RPE from eye cups of both Pde6b^{H620Q/H620Q}; Hif-2α^{t1Mcs}/Hif-2α^{tm1Mcs} and control the mice were isolated after incubation in trypsin at 37° C., 5% CO₂ for 45 min (Fernandez, et al. 2016), and analyzed by ERGs, as described in Example 1 and H&E staining as described in Example 2.

### Results

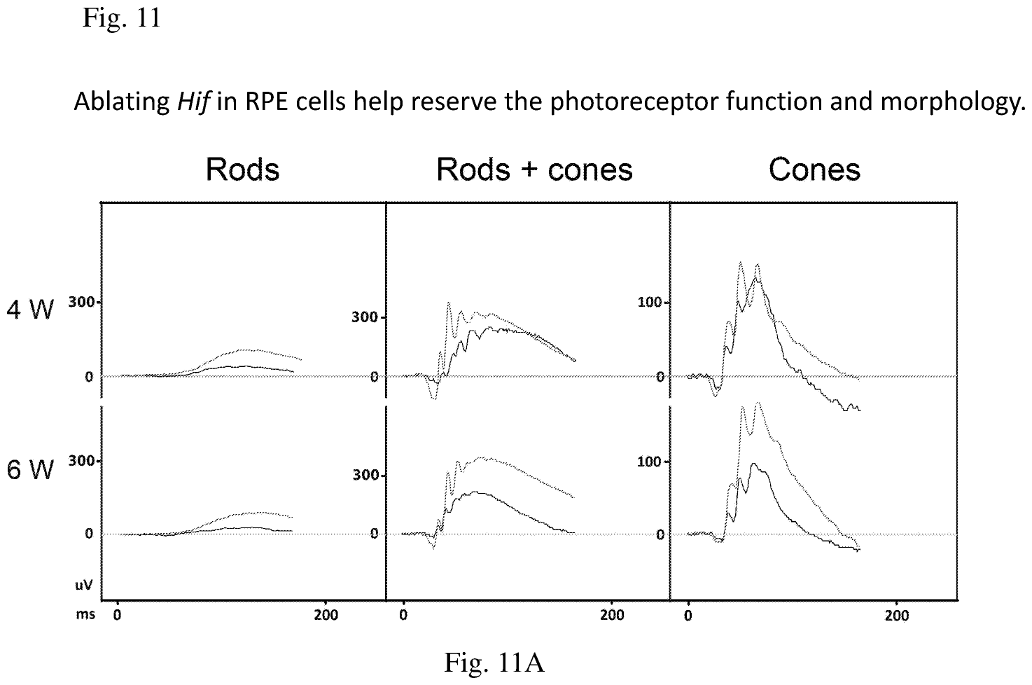

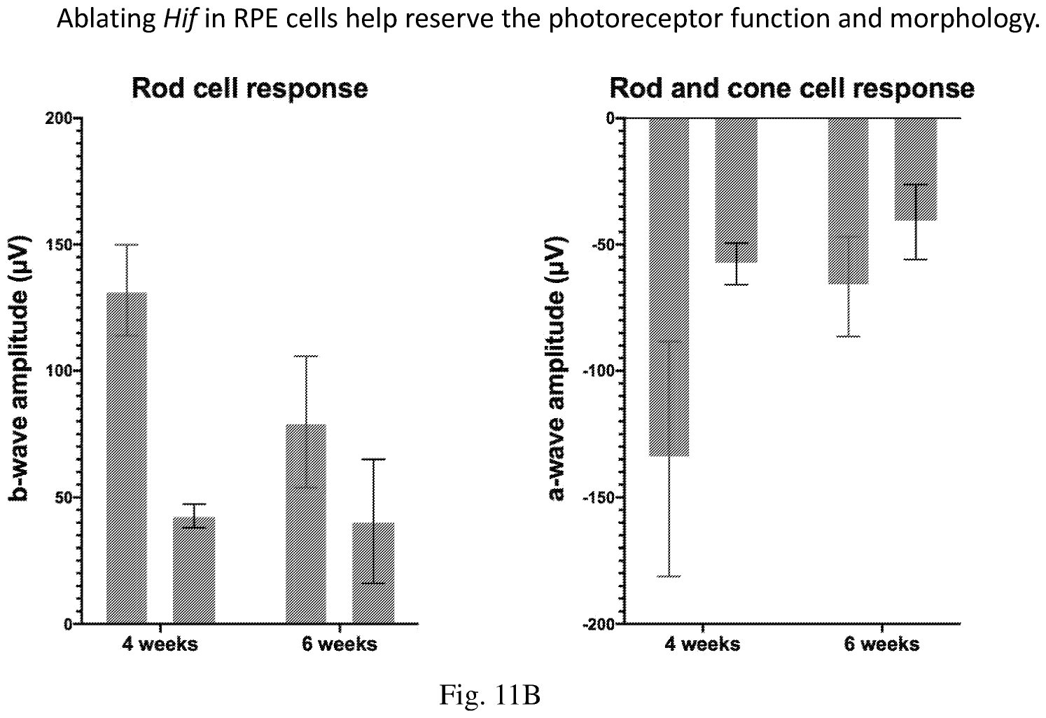

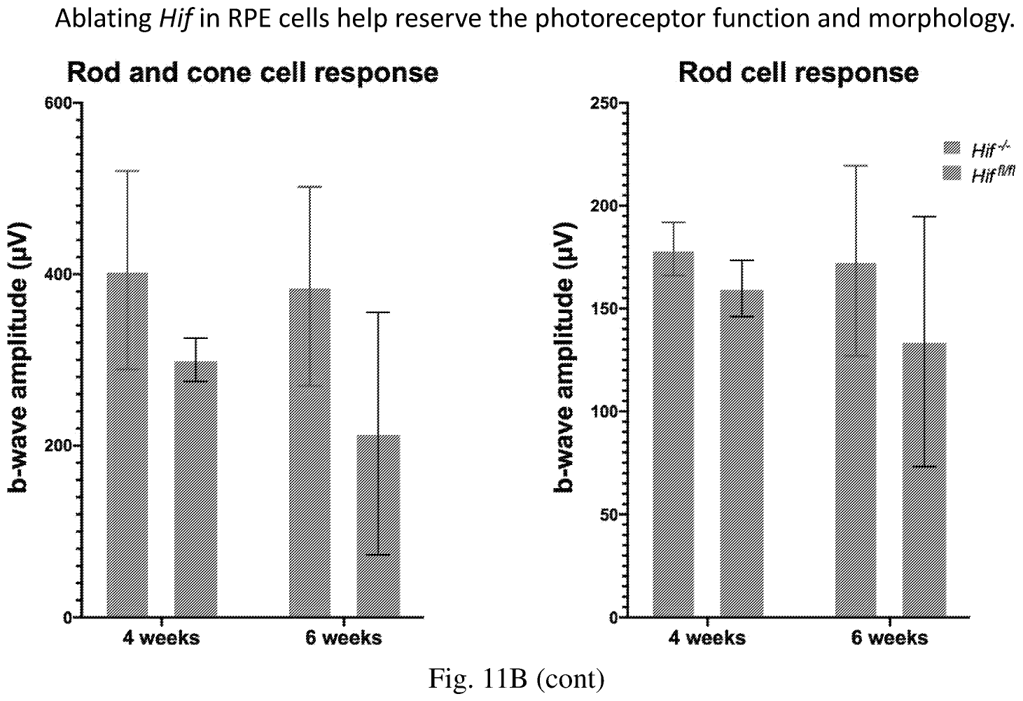

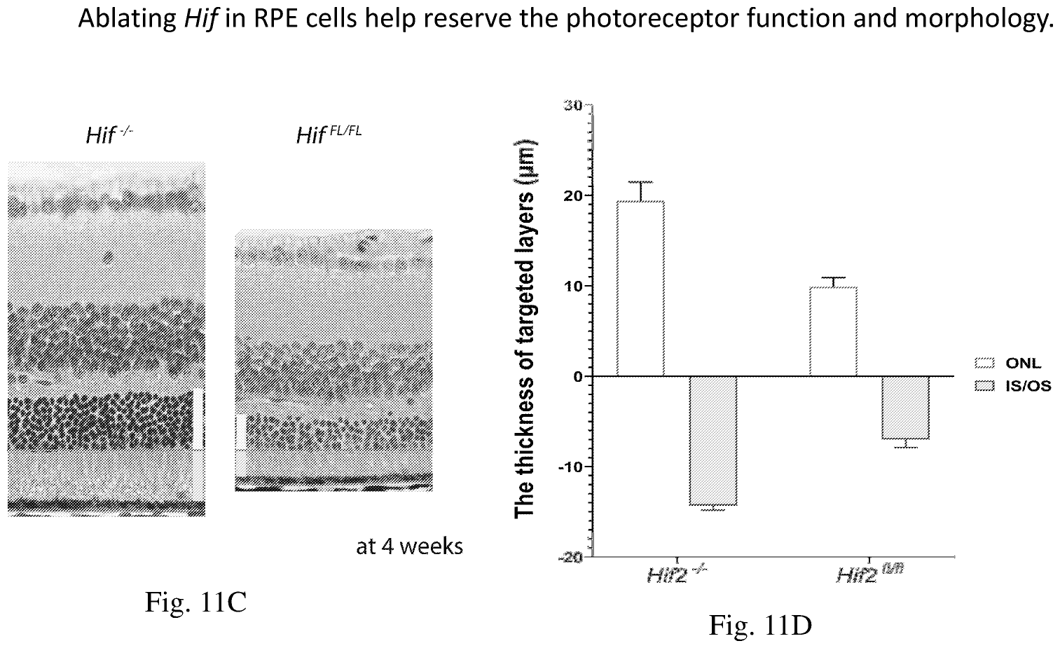

As shown in FIG. 11, ablating Hif2 specifically in RPE enhances electrophysiological function and survival in both rods and cones. After ablation Hif in RPE cells, the photoreceptor function was well preserved, including rod, rod+cone, and cone.

FIG. 11A shows ERG data obtained at 4 and 6 weeks under dark- and dark- and light-adapted conditions to acquire scotopic, photopic, and mixed rod-cone b-wave amplitudes (μV). Traces of the retinal function of the Hif^{-/-}Pde6b^{H620Q/H620Q} (red trace) with Hif2a ablated in RPE were shown at 4, 6 weeks comparing to the age match Hif^{doxP/loxP} Pde6b^{H620Q/H620Q} control (black trace).

FIG. 11B which quantifies the amplitude of the ERG traces, shows increased rod and cone cell responses in the Hif^{-/-}Pde6b^{H620Q/H620Q} mice.

Histology at four weeks shows thicker ONL and OS layers a greater width of photoreceptor outer nuclear (ONL) layer and IS/OS layer in the Hif^{-/-}Pde6b^{H620Q/H620Q} mice as compared to control mice. FIGS. 11C and 11D.

### Example 9—Targeted AAV Vector Delivered HIF Inhibitor to RPE Promoting Cone and Rod Survival

### Materials and Methods

The same materials and methods are used as in Example 1 for the RP mouse model, and subretinal injection and assessments including ERG, and immunohistochemistry and fluorescent staining with anti-arr antibody, and the experimental comparison.

As discussed, to increase OXPHOS and beta-oxidation in RPE, a VMD2 upstream region from −253 to +38 bp (approximately 300 bp) as an RPE-specific promoter (Esumi, et al. 2004) is used to deliver a gRNA inhibitor of HIF. Two vectors are generated: EIAV::U6-gRNAs_scramble;Vmd2::Cas9 as a control for validation and EIAV::U6-gRNAs_HIf2a; Vmd2::spCas9. The gRNAs with the sequences SEQ ID NOs: 1-4 are used in the vectors.

### Results

The ablation of HIF in RPE slows rod and cone degeneration, as is evident from ERG analysis and histology. Additionally, increase of OXPHOS and beta-oxidation in RPE collected from the injected eyes relative to noninjected eyes in the RP mouse model is also seen.

These results show that the targeted administration of an inhibitor of HIF to RPE decreased photoreceptor degeneration in a mouse model of RP.

### Example 10—Targeted Vector Delivered KEAP1 Inhibitor to RPE Promoting Cone and Rod Survival

### Materials and Methods

The same materials and methods are used as in Example 1 for the RP mouse model, and subretinal injection and assessments including ERG, and immunohistochemistry and fluorescent staining with anti-arr antibody, and the experimental comparison.

As discussed, to enhance antioxidant responses in RPE, a VMD2 upstream region from −253 to +38 bp (approximately 300 bp) as an RPE-specific promoter (Esumi, et al. 2004) is used to deliver dual gRNAs of KEAP1. Two vectors were generated: EIAV::U6-gRNAs_scramble, as a control for validation and EIAV::U6-gRNAs_KEAP1; Vmd2::spCas9 to test our hypothesis. The gRNA with SEQ ID NO: 5 is one gRNA used in the vector.

### Results

The ablation of KEAP1 in RPE slows rod and cone degeneration, as is evident from ERG analysis and histology. Additionally, the upregulation of ARE-related genes in RPE collected from the injected eyes relative to noninjected eyes in the RP mouse model is also seen.

These results show that the targeted administration of an inhibitor of KEAP1 to RPE decreased photoreceptor degeneration in a mouse model of RP.

### REFERENCES

Campochiaro and Mir. The mechanism of cone cell death in Retinitis Pigmentosa. Prog Retin Eye Res. 2018;62:24-37.

Chaffiol et al. A New Promoter Allows Optogenetic Vision Restoration with Enhanced Sensitivity in Macaque Retina. Mol Ther. 2017;25(11):2546-2560.

Chintalapudi et al. Isolation of Primary Murine Retinal Ganglion Cells (RGCs) by Flow Cytometry. J Vis Exp. 2017(125).

Chintalapudi et al. Isolation and Molecular Profiling of Primary Mouse Retinal Ganglion Cells: Comparison of Phenotypes from Healthy and Glaucomatous Retinas. Front Aging Neurosci. 2016;8:93.

Chou et al. High-Throughput Binocular Pattern Electroretinograms in the Mouse. Methods Mol Biol. 2018;1695:63-68.

Chrysostomou and Crowston. The photopic negative response of the mouse electroretinogram: reduction by acute elevation of intraocular pressure. Invest Ophthalmol Vis Sci. 2013;54(7):4691-4697.

da Cruz et al. Five-Year Safety and Performance Results from the Argus II Retinal Prosthesis System Clinical Trial. Ophthalmology. 2016;123(10):2248-2254. Davis et al. Functional rescue of degenerating photoreceptors in mice homozygous for a hypomorphic cGMP phosphodiesterase 6 b allele (Pde6bH620Q). Invest Ophthalmol Vis Sci. 2008 ;49(11):5067-5076.

Delettre et al. OPA1 (Kjer type) dominant optic atrophy: a novel mitochondrial disease. Mol Genet Metab. 2002 ;75(2):97-107.

Duncan et al. Inherited Retinal Degenerations: Current Landscape and Knowledge Gaps. Transl Vis Sci Technol. 2018; 7(4): 6.

Errijgers et al. FVB.129P2-Pde6b(+) Tyr(c-ch)/Ant, a sighted variant of the FVB/N mouse strain suitable for behavioral analysis. Genes Brain Behay. 2007;6(6):552-557.

Esumi et al. Analysis of the VMD2 promoter and implication of E-box binding factors in its regulation. J Biol Chem. 2004;279(18):19064-19073.

Fernandez-Godino et al. Isolation, culture and characterization of primary mouse RPE cells. Nat Protoc. 2016; 11(7): 1206-1218.

Fernandez-Marcos and Auwerx. Regulation of PGC-lalpha, a nodal regulator of mitochondrial biogenesis. Am J Clin Nutr. 2011; 93(4): 884S-890.

Guo et al. PGC-lalpha signaling coordinates susceptibility to metabolic and oxidative injury in the inner retina. Am J Pathol. 2014;184(4):1017-1029.

Hart et al. Genotype—phenotype correlation of mouse pde6b mutations. Invest. Ophthalmol. Vis. Sci. 2005 ;46(9):3443-50.

Harvey et al. Intravitreal injection of adeno-associated viral vectors results in the transduction of different types of retinal neurons in neonatal and adult rats: a comparison with lentiviral vectors. Mol Cell Neurosci. 2002;21(1):141-157.

Howell et al. Axons of retinal ganglion cells are insulted in the optic nerve early in DBA/2J glaucoma. J Cell Biol. 2007;179(7):1523-1537.

John et al. Essential iris atrophy, pigment dispersion, and glaucoma in DBA/2J mice. Invest Ophthalmol Vis Sci. 1998;39(6):951-962.

Larabee et al. Loss of Nrf2 exacerbates the visual deficits and optic neuritis elicited by experimental autoimmune encephalomyelitis. Mol Vis. 2016;22:1503-1513.

Lam et al. Leber hereditary optic neuropathy gene therapy clinical trial recruitment: year 1. Arch Ophthalmol. 2010; 128(9): 1129-1135.

Lee et al. N-Acetylcysteine promotes long-term survival of cones in a model of retinitis pigmentosa. J Cell Physiol. 2011; 226(7): 1843-1849.

MacVicar and Langer. OPA1 processing in cell death and disease—the long and short of it. J Cell Sci. 2016;129(12):2297-2306.

Malik et al. Accurate quantification of mouse mitochondrial DNA without co-amplification of nuclear mitochondrial insertion sequences. Mitochondrion. 2016;29:59-64.

Martin et al. Gene therapy with brain-derived neurotrophic factor as a protection: retinal ganglion cells in a rat glaucoma model. Invest Ophthalmol Vis Sci. 2003;44(10):4357-4365.

Mattapallil et al. The Rd8 mutation of the Crb 1 gene is present in vendor lines of C57BL/6N mice and embryonic stem cells, and confounds ocular induced mutant phenotypes. Invest Ophthalmol Vis Sci. 2012;53(6):2921-2927.

McMahon et al. Redox-regulated turnover of Nrf2 is determined by at least two separate protein domains, the redox-sensitive Neh2 degron and the redox-insensitive Neh6 degron. J Biol Chem. 2004; 279(30): 31556-31567.

Nickells et al. AAV2-Mediated Transduction of the Mouse Retina After Optic Nerve Injury. Invest Ophthalmol Vis Sci. 2017;58(14):6091-6104.

Porciatti. Electrophysiological assessment of retinal ganglion cell function. Exp Eye Res. 2015;141:164-170.

Punzo et al. Stimulation of the insulin/mTOR pathway delays cone death in a mouse model of retinitis pigmentosa. Nat Neurosci. 2009;12(1):44-52.

Quigley and Broman The number of people with glaucoma worldwide in 2010 and 2020. Br J Ophthalmol. 2006; 90(3):262-267.

Russell et al. Efficacy and safety of voretigene neparvovec (AAV2-hRPE65v2) in patients with RPE65-mediated inherited retinal dystrophy: a randomised, controlled, open-label, phase 3 trial. Lancet. 2017;390(10097):849-860.

Saleh et al. Longitudinal evaluation of retinal ganglion cell function and IOP in the DBA/2J mouse model of glaucoma. Invest Ophthalmol Vis Sci. 2007;48(10):4564-4572.

Takahashi et al. Gene therapy in inherited retinal degenerative diseases, a review. Ophthalmic Genet. 2018 ;39(5):560-568.

Tosi et al. Rapid and noninvasive imaging of retinal ganglion cells in live mouse models of glaucoma. Mol Imaging Biol. 2010;12(4):386-393.

Theodore et al. Multiple nuclear localization signals function in the nuclear import of the transcription factor Nrf2. J Biol Chem. 2008;283(14):8984-8994.

Wang et al. Varicosities of intraretinal ganglion cell axons in human and nonhuman primates. Invest Ophthalmol Vis Sci. 2003;44(1):2-9.

Wang et al. Transplantation of reprogrammed embryonic stem cells improves visual function in a mouse model for retinitis pigmentosa. Transplantation. 2010;89(8):911-919.

Wang et al. Origin of fundus hyperautofluorescent spots and their role in retinal degeneration in a mouse model of Goldmann-Favre syndrome. Disease models and mechanisms. 2013; 6(5): 1113-1122.

Wang et al. Deciphering primate retinal aging in single-cell resolution. Protein Cell 2020; https://doi.org/10.1007/s13238-020-00791-x

Williams et al. Vitamin B3 modulates mitochondrial vulnerability and prevents glaucoma in aged mice. Science. 2017;355(6326):756-760.

Xiong et al. NRF2 promotes neuronal survival in neurodegeneration and acute nerve damage. J Clin Invest. 2015;125(4):1433-1445.

Zhang et al. Genetic activation of Nrf2 protects against fasting-induced oxidative stress in livers of mice. PLoS One. 2013;8(3):e59122.

Zhang et al. Reprogramming metabolism by targeting sirtuin 6 attenuates retinal degeneration. J Clin Invest. 2016A;126(12):4659-4673.

Zhang, et al. Reprogramming towards anabolism impedes degeneration in a preclinical model fo retinitis pigmentosa. Human Mo. Genetic. 2016B 25(19), 4244-4255.

1-4. (canceled)

5. A method of delaying the onset of, treating, or preventing a neurodegenerative disease in a subject in need thereof, comprising administering to the subject a therapeutically effective amount of one or more viral vectors comprising a nucleic acid molecule encoding for one or more heterologous molecules which inhibits the expression of HIF or KEAP1, wherein the viral vector transduces retinal pigment epithelial (RPE) cells.

6. The method of claim 5, wherein the one or more heterologous molecules is selected from the group consisting of an antisense oligonucleotide, a small interfering RNA (siRNA), a short hairpin RNA (shRNA), a guide RNA (gRNA), an endonuclease, an aptamer, and combinations thereof.

7. The method of claim 5, wherein the viral vector further comprises an RPE cell specific promoter operatively linked to the one or more heterologous molecules.

8. The method of claim 7, wherein the RPE cell specific promoter is selected from the group consisting of VMD2 and RPE65.

9. The method of claim 6, wherein the one or more heterologous molecules is a gRNA which inhibits the expression of HIF.

10. The method of claim 6, wherein the one or more heterologous molecules is a gRNA which inhibits the expression of KEAP1.

11. The method of claim 5, wherein nucleic acid molecule encoding one or more heterologous molecules encodes for: a) at least one guide RNA that hybridizes to an endogenous HIF gene in the subject, and (b) an endonuclease; wherein the endonuclease cleaves the endogenous HIF gene creating a HIF knockout of the endogenous HIF gene in the subject, wherein the nucleic acid molecule is operatively linked to a RPE cell specific promoter.

12. (canceled)

13. The method of claim 11, wherein the viral vector is selected from the group consisting of adeno-associated viral (AAV) vector and lenti vector.

14. The method of claim 13, wherein the viral vector transduces RPE and the RPE cell specific promoter is selected from the group consisting of VMD2 and RPE65.

15. The method of claim 11, wherein the at least one guide RNA has a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and SEQ ID NO: 13.

16-20. (canceled)

21. A method of delaying the onset of, treating, or preventing a neurodegenerative disease in a subject in need thereof, comprising administering to the subject a therapeutically effective amount of one or more viral vectors comprising a nucleic acid molecule encoding for one or more heterologous molecules which inhibits the expression of KEAP1, wherein the viral vector transduces retinal ganglion cells (RGC).

22. The method of claim 21, wherein the one or more heterologous molecules is selected from the group consisting of an antisense oligonucleotide, a small interfering RNA (siRNA), a short hairpin RNA (shRNA), a guide RNA (gRNA), an endonuclease, an aptamer, and combinations thereof.

23. The method of claim 22, wherein the one or more heterologous molecules is a gRNA which inhibits the expression of KEAP1.

24. The method of claim 21, wherein the viral vector further comprises an RGC specific promoter operatively linked to the one or more heterologous molecules.

25. The method of claim 24, wherein the RGC specific promoter is selected from the group consisting of hSNCG and Pie (NEFL).

26. The method of claim 5, wherein the neurodegenerative disease is selected from the group consisting of glaucoma, retinitis pigmentosa (RP), age-related macular degeneration (AMD), autosomal dominant optic atrophy (ADOA), Alzheimer's disease, Parkinson's disease, Huntington's disease, Amyotrophic lateral sclerosis (ALS), and Lewy body dementia.

27. The method of claim 21, wherein the neurodegenerative disease is selected from the group consisting of glaucoma, retinitis pigmentosa (RP), age-related macular degeneration (AMD), autosomal dominant optic atrophy (ADOA), Alzheimer's disease, Parkinson's disease, Huntington's disease, Amyotrophic lateral sclerosis (ALS), and Lewy body dementia.

28. The method of claim 5, wherein the viral vector is selected from the group consisting of an adeno-associated viral (AAV) vector and lenti vector.

29. The method of claim 21, wherein the viral vector is selected from the group consisting of an adeno-associated viral (AAV) vector and lenti vector.

30. The method of claim 5, wherein the nucleic acid molecule inhibits or targets HIF.

31. The method of claim 30, wherein the HIF is HIF2-alpha.

32. The method of claim 9, wherein the gRNA has a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and SEQ ID NO: 13.

33. The method of claim 10, wherein the gRNA has a sequence selected from the group consisting of SEQ ID NO: 14, SEQ ID NO: 15, SEQ ID NO: 16, SEQ ID NO: 17, SEQ ID NO: 18, SEQ ID NO: 19, and SEQ ID NO: 20.

34. The method of claim 23, wherein the gRNA has a sequence selected from the group consisting of SEQ ID NO: 14, SEQ ID NO: 15, SEQ ID NO: 16, SEQ ID NO: 17, SEQ ID NO: 18, SEQ ID NO: 19, and SEQ ID NO: 20.

35. The method of claim 11, wherein the endonuclease is a Cas nuclease.

36. The method of claim 11, wherein the HIF gene is HIF2-alpha.
